(19) United States
(12) Patent Application Publication
(10) Pub. No.: US 2012/0269834 A1
(43) Pub. Date: Oct. 25, 2012

(54) IMMUNOGLOBULIN CLEAVAGE FRAGMENT VACCINE COMPOSITIONS

(76) Inventors: Robert Jordan, Radnor, PA (US); David M. Knight, Berwyn, PA (US); Randall Brezski, Radnor, PA (US)

(21) Appl. No.: 13/318,168

(22) PCT Filed: Nov. 19, 2010
(86) PCT No.: PCT/US10/57396

§ 371 Date: Oct. 31, 2011

(51) Int. Cl. A61K 39/00; A61P 9/00; A61P 9/04; A61P 29/00; A61P 35/00; A61P 31/04; A61K 39/385; A61P 33/00

(52) U.S. Cl. 424/185.1; 424/184.1

(57) ABSTRACT

The invention relates to methods and reagents for the preparation and use of a therapeutic immunospecific for IgG breakdown products retaining antigen binding but having lost effector functions. The reagents of the invention may be used as immunogens for the purpose of prophylactic or therapeutic vaccination of a human subject.

### BACKGROUND OF THE INVENTION

1. Field of the Invention

The invention relates to methods, compositions, and uses of immunoglobulins reactive with a target specific natural or therapeutic immunoglobulin, where the binding will restore bioactivity inherent in the cleaved and bound fragment of the targeted natural or therapeutic immunoglobulin, thereby providing or reconstituting the bioactivity. The invention further provides immunogenic immunoglobulin fragments for use in a vaccine for the production of such an immunoglobulin ex vivo or in vivo.

2. Description of the Related Art

Circulating immunoglobulins, and specifically those antibodies of the IgG class, are major serum proteins. It is well-known that human proteases are associated with inflammatory, proliferative, metastatic, and infectious diseases. Human proteases such as matrix metalloproteinases (MMPs) and neutrophil elastase cleave the IgGs heavy chain polypeptide at a residue unique to each protesase as do bacterial proteases such as glutamyl endopeptidase (Staph. aureus) or immunoglobulin degrading enzyme of streptococcus (Strep. pyogenes). The cleavage sites in the heavy chain are clustered around the region termed the hinge domain, where the interchain disulfide linkage of the two heavy chains occurs. The region below the hinge constitutes the Fc region and comprises binding sites responsible for the effector functions of IgG. In the case of microorganisms, protease expression is a potential adjunctive virulence pathway allowing organisms to avoid opsonization (Rooijakkers et al. Microbes and Infection 7: 476-484, 2005) in so far as the proteolytic release of the Fc domain by cleavage below the hinge effectively neutralizes functions that would otherwise lead to the targeting and killing of that pathological cell. Thus, the elaboration of specific proteases may be representative of a myriad of diseases states including cancer, inflammation and infectious diseases.

IgG degradation is enhanced in pathologic in vivo environments as evidenced by the presence of natural IgG autoantibodies that bind to the cleaved hinge domain (Knight et al., 1995; Nasu et al., 1980; Persselin and Stevens, 1985, Terness, et al. 1995 J Imunol. 154: 6446-6452). These autoantibodies also bind the Fab and F(ab′)₂ fragments generated by several proteinases (including papain and pepsin), with particularly strong reactivity to the lower hinge domain remaining as C-terminal residues in F(ab′)2 molecules (Terness et al., 1995). The detection of the actual cleavage products have been reported (Fick et al., 1985; Goldberg and Whitehouse, 1970; Waller, 1974) but a robust assay which would allow these fragments to serve as biomarkers has not been developed possibly due to the low concentrations in serum resulting from rapid clearance of the various fragments or to technical problems in detecting the fragments amidst the large amount of intact immunoglobulin in blood and tissues. A specific antibody was prepared (Eckle, et al. 1988. Adv. Exp. Med. Biol. 240: 531-534) for detection of human neutrophil elastase cleaved Fc domain which detected Fc at a median concentration of 0.62 ug/ml directly in synovial fluid of rheumatoid arthritis patients but not in synovial fluid from patients with other types of joint disease.

The ability to correct for the loss of IgG function in vivo and compensate for the process of IgG cleavage in the body of subjects suffering from protease specific disease activity has not been heretofore envisioned as a therapeutic strategy.

### SUMMARY OF THE INVENTION

The invention relates to antibodies and vaccines for producing antibodies, which are specific for IgG cleavage products which result from the release of proteolytic enzymes capable of degrading target bound or unbound IgG. The antibodies and vaccines are useful for restoring effector functions to a therapeutic IgG antibody composition which has been subject to protease degradation.

In another embodiment of the invention, disease specific cleavage site peptides representing the newly created C-terminal sequence of an IgG cleavage product are provided. These peptides are also useful for immunizing, panning, and selection of the anti-IgG cleavage product antibody of the invention. In one aspect, the peptide is selected from the group consisting of at least five (5) contiguous amino acids selected from the human IgG hinge region sequences of SEQ ID NO: 1, 2, 3, or 4 that are on the amino terminal side of a protease cleavage site. In one embodiment, the polypeptides are selected from those of SEQ ID NOs. 5-11 and N-terminal truncations thereof In another aspect, a method of designing a peptide immunogen based on the proteolytic cleavage site of a human IgG molecule is provided.

In one embodiment of the invention, methods of preparation of an anti-IgG cleavage product antibody of the invention are provided including nucleic acid sequences, vectors, and host cells for the recombinant production of anti-IgG cleavage product antibodies. In another aspect of the method of manufacturing the anti-IgG cleavage product antibodies, immunized host animals are provided which animals provide an antibody of the invention. In a particular embodiment, the animal is a human and the anti-IgG cleavage product is generated by administration of a cleavage site peptide immunogen selected from the group consisting of at least five (5) contiguous amino acids selected from the human IgG hinge region sequences of SEQ ID NO: 1, 2, 3, or 4 that are on the amino terminal side of a protease cleavage site such as the sequences of SEQ ID NOs. 5-11 and N terminal truncations thereof

In another embodiment of the invention, a kit for detection of anti-IgG cleavage product is provided comprising anti-IgG cleavage product antibodies of the invention for use in diagnosis or monitoring a disease characterized by the production of IgG cleaving proteases.

A further embodiment of the invention, is a method of administering an anti-IgG cleavage specific antibody to a patient as a method of treatment, thereby restoring effector functions to a therapeutic antibody composition which has been subjected to protease degradation. In accordance with the method, effector function is restored to the IgG cleavage product by administering the antibodies of the present invention which specifically bind to the IgG cleavage product.

In another aspect of the invention, a human suffering from a disease characterized by the elaboration of disease specific proteases, can be treated by administration of a cleavage site specific peptide immunogen selected from the group consisting of at least five (5) contiguous amino acids of the human IgG hinge region sequences of selected from SEQ ID NO: 1, 2, 3, or 4; and that are positions on the amino (N) terminal side of a protease cleavage site such as the sequences of SEQ ID NOs. 5-11 and N-terminal truncations, species homologs and chemical homologs thereof, to restore effector functions of IgG cleavage products in such patient. In a specific embodiment, the cleavage site specific peptide immunogen is represented by a peptide fragment that is N-terminal to the cleaved human IgG1 terminating with amino acid Glu233 (EU numbering), and the disease is selected from a S. aureaus infection characterized by the release of glutamyl endopeptidase I (GluV8), or neutrophilic release of cathepsin G. In another embodiment, the the cleavage site specific peptide immunogen is represented by a peptide fragment N-terminal cleaved human IgG1 terminating with amino acid Pro232 (EU numbering), and the disease characterized by a form of human cancer characterized by the release of MMP-3, MMP-12. In another embodiment, the cleavage site specific peptide immunogen is represented by a peptide fragment N-terminal to the IdeS of Streptococcus pyogenes cleaved human IgG1 terminating at Gly236 (EU numbering), and the disease is a Streptococcus spp infection.

In another embodiment of the invention, a kit is provided comprising anti-IgG cleavage product antibodies of the invention and instructions for use.

### BRIEF DESCRIPTION OF THE SEVERAL VIEWS OF THE DRAWING

FIG. 1 depicts the various domains of a typical mammalian IgG class antibody showing their relationship to the hinge and the pepsin and papain cleavage products defined as Fab, F(ab′)₂, and Fc.

FIG. 2 shows the sequence of the human IgG1 heavy chain around the hinge region; the positions of major proteolytic cleavages are indicated by arrows.

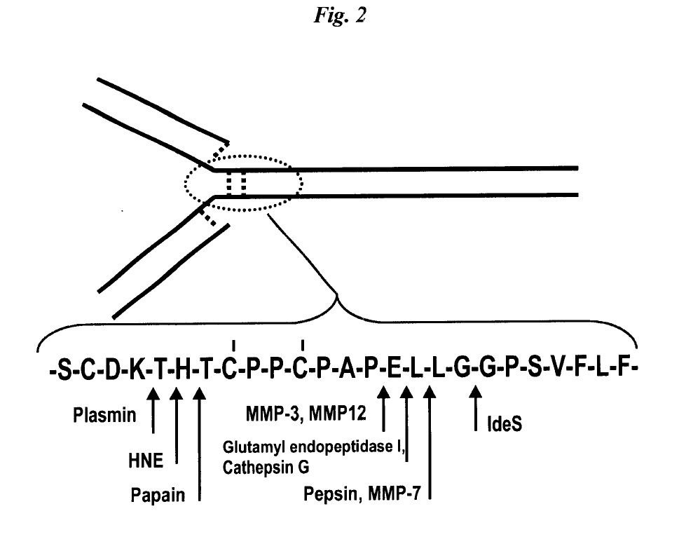

FIG. 3 is a graph showing the relative specificity of the antisera generated in rabbits immunized with fragments of human IgG enzymatically generated from three different proteases: MMP-3, V8, and IdeS;. Rabbits were immunized with conjugated peptides having the sequences TCPPCPAP, residues 7-14 of SEQ ID NO: 1 corresponding to the MMP-3 cleavage site; TCPPCPAPE, residues 7-15 of SEQ ID NO: 1 corresponding to the glutamyl endopeptidase site; and TCPPCPAPELLG, residues 7-18 of SEQ ID NO: 1 corresponding to the IdeS site. ELISA reactivity of three individual rabbit polyclonal anti-cleavage site specific peptide antibody preparations were tested for their ability to bind with F(ab′)₂ fragments of Mab3 IgG1κ, as well as ScIgG and intact IgG. The F(ab′)₂ fragments were generated with human recombinant MMP-3, staphylococcal glutamyl endopeptidase I and recombinant IdeS from Strep. pyogenes. The antibody preparation showed binding to scIgG and F(ab′)₂ but not to intact IgG. Bars correspond to the mean±standard deviation of three replicate wells.

FIG. 4 is a Western blot showing the reactivity of rabbit polyclonal antibody preparations with antibody digests: Mab3 human IgG1 intact or that had been partially digested with MMP-3, glutamyl endopeptidase (V8) or IdeS was separated by SDS-PAGE followed by immunoblotting, where (A) was blotted with anti-human IgG (H+L) [lanes 1-4] or anti . . . LLG rabbit polyclonal [lanes 6-10]. (B) was blotted with anti . . . PAP [lanes 2-5] or anti . . . APE [lanes7-10]. The blots were cut prior to incubation with antibodies through lane 5 in panel A and lane 6 in panel B to allow for detection with the individual antisera.

FIG. 5 is a western blot developed using the RAH-1 reagent on samples from an analysis of IgG degradation in the synovial fluid from 5 RA patients and compared to samples from in vitro proteolytic digests of a monoclonal IgG1 with MMP-3, glutamyl endopeptidase (V8) and IdeS.

FIG. 6 is a dot plot of the individual values of scIgG detected by the reagent RAH-1 in human serum samples from patients diagnosed with the diseases as indicated as compared to that in a group of normal human serum samples where the lines indicated mean values in each group.

FIG. 7 shows the relative reactivity of rabbit monoclonal antibody targeting cleavage fragments from human IgG1 hinge as peptide analogues and to antibody fragments terminating at the residue specified (see FIG. 3).

FIG. 8 shows the concentration dependence of three different rabbit monoclonal antibodies targeting cleavage fragments of human IgG1 hinge in restoring complement-dependent cell lysis (CDC) to F(ab′)₂ created by digesting IgG1 with IdeS compared to a rabbit polyclonal prepared to cleavage peptide analogues (rb poly).

FIG. 9 shows the mean log colony bacterial count and SEM in the whiffle ball after inoculation with Staph. aureaus of control rabbits (black line) vs immunized rabbits (dashed line).

FIG. 10 is Kaplan Meier survival plot for rabbits inoculated with Staph. aureaus showing the control rabbits (black line) and immunized rabbits (dashed line).

FIG. 11 shows the change in tumor volume over time for five groups of mice implanted with human breast carcinoma cells (MDA-MB231) and treated with either a tumor targeted Mab (860) alone (solid triangles); a protease treated preparation of 860, 860 scIgG (closed circles); the anti-hinge antibody preparation 2095-2; the 860 scIgG plus the anti-hinge antibody preparation 2095-2; or PBS (open circles).

FIG. 12 shows the effect on circulating platelet numbers of an anti-platelet integrin Mab (c7E3) or proteolytic cleavage products of the Mab, administered alone and in combination with anti-cleavage site mAb, 2095-2, on platelet counts in dogs, where each point represents the mean±SD of the determinations of three animals.

BRIEF DESCRIPTION OF THE SEQUENCE LISTING

| SEQ | |
|---|---|
| ID | |
| NO: | DESCRIPTION |
| 1 | Human IgG1 hinge region |
| | SCDKTHTCPP CPAPELLGGP SVFLF |
| 2 | Human IgG4 hinge region |
| | TCNVDHKPSN TKVDKRVESK YGPPCPSCPA |
| | PEFLGGPSVF LF |
| 3 | Human IgG2 hinge region |
| | TCNVDHKPSN TKVDKTVERK CCVECPPCPA |
| | PPVAGPSVFL F |
| 4 | Human IgG3 hinge region |
| | SCDTPPPCPR CPEPKSCDTP PPCPRCPAPE |
| | LLGGPSVFLF |
| 5 | MMP-3 and MMP12 cleavage peptide |
| 6 | Glutamyl endopeptidase 1 and |
| | Cathepsin G cleavage peptide |
| 7 | IdeS cleavage peptide |
| 8 | Plasmin cleavage peptide |
| 9 | HNE cleavage peptide |
| 10 | Pepsin and MMP-7 cleavage peptide |
| 11 | Papain cleavage peptide |
| 12 | IdeS analogueue cleavage site peptide |
| | immunogen, TAPPAPAPELLG |
| 13 | IdeS analogueue cleavage site peptide, |
| | TSPPSPAPELLG |
| 14 | IdeS false analogueue cleavage site |
| | peptide, TSPPSPAPALLG |
| 15 | IdeS false analogueue cleavage site |
| | peptide, TSPPSPAPEALG |
| 16 | Glutamyl endopeptidase cleavage site |
| | analogueue peptide, CTSPPSPSPAPE |

### DETAILED DESCRIPTION OF THE INVENTION

### Abbreviations

Abs=antibodies, ADCC=antibody-dependent cell-mediated cytotoxicity; CDC=complement directed cytoxicity; HNE=human neutrophil elastase; IdeS=immunoglobulin degrading enzyme of S. pyrogenes; Ig=immunoglobulin; Mab=monoclonal antibody; MMP=matrix metalloprotease or metalloproteinase; N-terminal=amino terminal; scIgG=single cleaved IgG; SA=streptavidin; GluV8=glutamyl endopeptidase I from Staph. aureus.

### Definitions

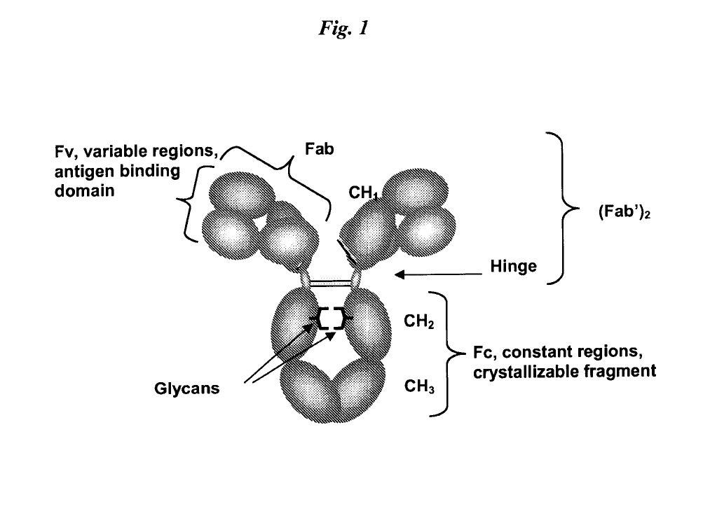

Antibody fragments; Fab, F(ab′)₂, and Fc are terms describing proteolytic cleavage products of IgG antibodies which may be further dissociated by reduction of the disulfide bonds between the heavy chains (the core hinge region). Classic proteolytically generated antibody fragments, include: Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), where reduction removes the disulfide linkage between cysteine residues forming interchain linkages (refer to FIG. 1). As the Fc fragment was described as a papain cleavage fragment and which cleaves human IgG1 at residue 224 (EU numbering), which is N-terminal to the hinge, the Fc fragment is assumed to retain the hinge and the disulfide linkages between the heavy chains, however, due to the high degree of association between the heavy chain CH2-CH3 dimers in the antibody, a dimeric structure is retained even in the absence of the disulfide (hinge) bonds. Thus, as used herein “Fc” refers to the dimeric structure formed by association of the heavy chain CH2-CH3 segments whether covalently bound or not. It will be understood that the non-covalently associated Fc may be distinguished from the disulfide linked Fc by its ability to undergo dissociation into CH2-CH3 monomers in the presence of a denaturant such as a detergent.

Human antibodies are immunoglobulins which basic structure is a dimer of two heterodimers, where the heterodimers are each comprised of a heavy and a light chain polypeptide.

The terms terms “proteolytic”, “protease”, “proteinase” and “proteolytic enzyme” are used interchangeably and mean an agent, e.g. enzyme, which is able to cleave a polypeptide chain producing two or more fragments, where the enzyme acts under normal temperature and under physiological conditions or physiologically compatible conditions. Physiological conditions include any temperature, buffer, cation, anion, substrate, catalyst, pH, cofactor, or the like which is a naturally found in the body of a living mammal whether in health or disease. However, the protease may be derived from a non-mammalian source such as from a pathogen which may be of any type of life form. Proteases are hydrolases that act on peptide bonds.

By “scIgG” or “single cleaved IgG” is meant any immunoglobulin class G molecules having a heterodimeric structure comprising two heavy chains and two light chains, where one of the heavy chains has been subjected to proteolytic cleavage on a single heavy chain while the second heavy chain remains intact.

By “upstream” relative to an amino acid sequence written from the N-terminal to the C-terminal residue is meant the residues in the sequence towards the N-terminus from a given residue. Conversely, by “downstream” relative to an amino acid sequence is meant the residues in the sequence towards the C-terminus from a given residue.

### Antibody Functions by Substructure

In general, immunoglobulins, antibodies, consist of regions of continuous polypeptide chain comprising approximately 100 amino acids, and each about 10-11 kDa, which show a characteristically folded globular domain and represent different elements of the structure. For immunogammaglobulins are antibodies (IgGs) which are proteins of about 150 kDa in which these domains are grouped together into segments; the Fab segment is comprised of a light chain variable joined to a light chain constant region in a single chain linked through a disulfide bond to the heavy chain first constant region (CH1) which is contiguous with the heavy chain variable region; Fc is comprised of two contiguous heavy chain constant regions (CH2 and CH3) linked through two or three disulfide bonds in the hinge region. Studies have shown that proteases, such as papain and pepsin, preferentially cleave antibodies at sites which are between the segments. Two identical Fab segments connected via the hinge region to one Fc segment, thus form a Y-shaped conformation of the 150 kDa structure (see FIG. 1). Fab segments generated using papain typically have a molecular weight of 46 kDa, nonreduced F(ab′)₂ typically have a molecular weight of 90-100 kDa, and nonglycosylated, nonreduced Fc will have an apparent molecular weight of approximately 50-60 kDa. However, as each antibody species, and each subclass of antibody within a species, is slightly different, the exact nature and location of the cleavage and cleavage products are variant.

Antigen binds to antibodies via an antigen binding site in the variable domains of each pair of light and heavy chains (FIG. 1). Other molecules, known as effector molecules or cells, bind to other sites in the remainder of the molecule, i.e. other than the antigen binding sites, and this portion of antibody includes the more invariant immunoglobulin sequences, “the constant portion” of an antibody, such sites being located particularly in the Fc region constituted by the portions of the heavy chains extending beyond the ends of the light chains: the upper hinge, lower hinge, CH2 and CH3 domains.

Antibodies have several effector functions mediated by binding of effector molecules. For example, binding of the C1 component of complement to antibodies activates the complement system. Activation of complement is important in the opsonisation and lysis of cell pathogens (a process called complement-mediated cytotoxicity or CDC). The activation of complement stimulates the inflammatory response and may also be involved in autoimmune hypersensitivity. Further, antibodies bind to cells via the Fc region, with a Fc receptor site on the antibody Fc region binding to a Fc receptor (FcR) on a cell. There are a number of Fc receptors which are specific for different classes of antibody, including IgG (gamma receptors), IgE (eta receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, complement-directed cytotoxicity, or ADCC), release of inflammatory mediators, placental transfer and endothelial cell reuptake (via neotal Fc receptor or FcRn) and control of immunoglobulin production.

The sequences around the hinge domain are conserved among IgG isotypes (SEQ ID NO: 1-4) and among mammalian species generally. The IgG1 (SEQ ID NO: 1) and IgG3 (SEQ ID NO: 1) isotype comprise a Leu-Leu pair that is a structural motif for binding to Fcy receptor(s) and for Fc effector functions. Other residues downstream of the “hinge core” which typically comprises at least one cysteine separated by two non-cysteine residues, are also conserved.

A cleavage product, single cleaved or scIgG, of human IgG 1 is formed by human and bacterial proteases when proteolysis occurs on one of the two heavy chain polypeptides that comprise an IgG, while not disrupting the overall composition of the heterodimeric molecule as noted previously (Gearing A J H et al, Immunol. Lett. 81: 41-48, 2002; Vincents et al, Biochemistry 43: 15540-15549, 2004). The scIgG is likely the more abundant product of in vivo proteolysis found in the serum than are other fragments.

The kinetics of proteolytic cleavage under physiological conditions lead to a larger proportion of proteolytically cleaved IgG being in the scIgG conformation than species which are products of multiply cleavage events, such as the F(ab′)₂ (FIG. 1) (U.S. patent Ser. No. 12/185,333 filed 4 Aug. 2008). In the process of testing a large number of proteases, it was determined that the first cleavage of a heavy chain constant region in an intact IgG proceeds more rapidly than the second, a sequence that leads to a temporal accumulation of the singly cleaved species. This single cleaved version of the IgG molecule is indistinguishable from its intact parent in many ways (e.g. molecular size, antigen binding, ability to be recognized by protein A/G).

Antibodies capable of specific binding of proteolytic cleavage products including F(ab′)₂ and scIgG were generated using cleavage site analogue peptides of the invention, which recognize human IgG1 cleavage products but do not recognize intact IgG. The co-pending patent application further discloses that antibody preparations can be selected that specifically recognize IgG cleavage products retaining antigen binding specificity and can restore effector functions such as CDC and ADCC to the cleaved IgG.

### Proteolytic Enzymes and Disease Association

The applicants demonstrated that antibody cleavage products, including scIgGs, similar in size to those generated with in vitro enzyme panel, are detectable in inflammatory exudates such as synovial fluid from patients with rheumatoid arthritis. Further, scIgG can be detected in the serum of patients with a number of diseases in which localized proteolytic activity is a known characteristic of the pathology. The scIgG in these disease states is at higher concentrations than in healthy normal volunteers and is also higher than in the serum of patients with less severely inflammatory disease.

Table 1 is a summary of the findings concerning the action of human proteases and two bacterial proteases on human IgG1. The term “Coagulation proteinases” refers to serum coagulation cascade proteins including; FXIIa, FIXa, FXa, thrombin and activated protein C; plasmin was plasminogen co-incubated with plasminogen activators; tPA, streptokinase and staphylokinase; “plasminogen activators alone” are without plasminogen; and the MMPs were recombinant proteinases obtained either as the active form or the pro-enzyme as detailed in the Materials; and “None” denotes no detectable cleavage in 24 hours. Except where indicated all enzymes where human. The residue designations are for the EU numbering system for the IgG1 antibody heavy chain where the 25 residues of SEQ ID NO: 1 corresponds to residues 219 through 243 of the complete mature heavy chain.

TABLE 1

| | | | Disease | | |
|---|---|---|---|---|---|
| | | Proteinase | Association | Cleaved | Major |
| Enzyme | Source | Type | (Ref) | Site | Product |
| Cathepsin G | Human | Serine | Emphysema, | Glu²³³- | F(ab′)₂ + |
| | Neutrophil | endopeptidase | IPF, RA (2, 3) | leu²³⁴ | Fc |
| | granules | | | | |
| Cathepsin B | Human | Serine | | | None |
| | Neutrophil | endopeptidase | | | |
| | granules | | | | |
| Cathepsin D | Human | Serine | | | None |
| | Neutrophil | endopeptidase | | | |
| | granules | | | | |
| Neutrophil | Human | Serine | Amyloidosis, | Thr²²³- | Fab + Fc |
| elastase | Neutrophil | endopeptidase | lung emphysema, | his²²⁴ | |
| (HNE, leukocyte | granules | | cystic fibrosis, | | |
| elastase, PMN | neutrophils | | ARDS, RA, tumor | | |
| elastase) | | | invasion (2, 3) | | |
| Pancreatic | | | Pancreatititis | | |
| elastase | | | (3) | | |
| Proteinase 3 | Human | Serine | | | None |
| (myeloblastin) | Neutrophil | endopeptidase | | | |
| | granules | | | | |
| Tryptase | Human | Serine | Anaphylaxis, | | None |
| | Neutrophil | endopeptidase | fibrosis (2) | | |
| | granules | | | | |
| | mast cells | | | | |
| Chymase | Human | Serine | Inflammation, | | None |
| | Neutrophil | endopeptidase | cardiovascular | | |
| | granules | | diseases (2, 3) | | |
| | mast cells | | | | |
| Kallekrein | Human | Serine | | | None |
| | Neutrophil | endopeptidase | | | |
| | granules | | | | |
| Coagulation | Human | Serine | | | None |
| proteinases | Neutrophil | endopeptidase | | | |
| | granules | | | | |
| Plasmin | Human | Serine | Cell migration | Lys²²³- | Fab + Fc |
| (fibrinolysin) | Neutrophil | endopeptidase | (e.g. tumors)(2) | thr²²⁴ | |
| | granules | | Streptococcal | | |
| | | | infection (6) | | |
| Plasminogen | Human | Serine | | | None |
| activators | Neutrophil | endopeptidase | | | |
| alone | granules | | | | |
| Interstitial | Human | Metalloendo- | RA, OA, IBD, | | None |
| collagenase | (fibroblasts, | peptidase | IPF, aneurysms | | |
| (MMP-1) | chondrocytes, | | (1) | | |
| Stromelysin | Human | Serine | RA, OA, | Pro²³²- | F(ab′)₂ + |
| (MMP-3) | Neutrophil | endopeptidase | atherosclerotic | glu²³³ | Fc |
| | granules | | plaque, Crohn's | | |
| | fibroblasts, | | disease, colitis, | | |
| | chondrocytes, | | some tumors (1, 4) | | |
| | osteoclasts, | | | | |
| | macrophages | | | | |
| Matrilysin | Human | Serine | Invasive tumors | Leu²³⁴- | F(ab′)₂ + |
| (MMP-7) | Neutrophil | endopeptidase | (1, 4) | leu²³⁵ | Fc |
| | granules | | | | |
| | glandular | | | | |
| | epithelial | | | | |
| | cells | | | | |
| Collagenase 2 | Human | | Inflammation, | | None |
| (MMP-8) | Neutrophil | | RA, OA (1, 4) | | |
| | granules | | | | |
| | neutrophils | | | | |
| Macrophage | Human | Serine | Inflammation, | Pro²³²- | F(ab′)₂ + |
| metalloelastase | Neutrophil | endopeptidase | tissue | glu²³³ | Fc |
| (MMP-12) | granules | | destruction | | |
| | macrophages | | when over- | | |
| | | | expressed, | | |
| | | | aneurysms, | | |
| | | | atherosclerotic | | |
| | | | plaque (1) | | |
| Cathepsin S | Human | Cysteine | | | None |
| | Neutrophil | endopeptidase | | | |
| | granules | | | | |
| Glutamyl | Staph. | Serine | Staph. Aureus | Glu²³³- | F(ab′)₂ + |
| endopeptidase | aureus | endopeptidase | infection (2) | leu²³⁴ | Fc |
| I (Glu V8 | | | | | |
| protease) | | | | | |
| Immunoglobulin | Strep. | Serine | Strep. Pyogenes | Gly²³⁶- | F(ab′)₂ + |
| degrading | Pyogenes | endopeptidase | infection (5) | gly²³⁷ | Fc |
| Enzyme of | | | | | |
| Streptococcus | | | | | |
| (IdeS) | | | | | |

(1) Barrett A. J., Rawlings N. D. and Woessner J. F. (Eds.), Handbook of Proteolytic Enzymes Vol. 1, Elsevier, Amsterdam, 2004.
(2) Barrett A. J., Rawlings N. D. and Woessner J. F. (Eds.), Handbook of Proteolytic Enzymes Vol. 2, Elsevier, Amsterdam, 2004.
(3) Powers, J C.. “Proteolytic Enzymes and Disease Treatment” 1982. In: Feeney and Whitaker (eds). Modification of Proteins: Food, Nutritional, and Pharmacological Aspects. Advances in Chemistry Series 198. ACS, Washington, D.C. 1982 pp 347-367.
(4) Tchetverikov I., Ronday H. K., van El B., Kiers G. H., Verzijl N., TeKoppele J. M., Huizinga T. W. J., DeGroot J. and Hannemaaijer R., 2004. MMP Profile in paired serum and synovial fluid samples of patients with rheumatoid arthritis. Ann. Rheum. Dis. 63, 881-883.
(5) Vincents B., von Pawel-Rammingen U., Björck L. and Abrahamson M., 2004. Enzymatic characterization of the streptococcal endopeptidase, IdeS, reveals that it is a cysteine protease with strict specificity for IgG cleavage due to exosite binding. Biochemistry 43, 15540-15549.
(6) Sun H., Ringdahl U., Homeister J. W., Fay W. P., Engleberg N. C., Yang A. Y., Rozek L. S., Wang X., Sjobring U., Ginsburg D., 2004. Plasminogen is a critical host pathogenicity factor for group A streptococcal infection. Science. 305, 1283-1286.

The detection of cleaved IgG including scIgG was accomplished by the generation of affinity-purified polyclonal antibodies (rabbit) that specifically bind to newly exposed epitopes in the cleaved heavy chain at or around the hinge disulfides, but do not react with the intact, non-cleaved IgG molecule. Confirmatory support for the detection of scIgG in serum is its prolonged circulating lifespan similar to intact IgG. The ability to detect scIgG in the bodily fluids or blood of diseased individuals is a potentially novel biomarker strategy.

### Antibody Preparations

An antibody for having specifity for a cleavage site specific peptide for used as a diagnostic, prophylactic or therapeutic agent for use in the methods of the invention can be prepared in several ways well known in the art using criteria, methods, and immunogens as exemplified herein to raise or select antibodies useful in the practice of the invention.

In one aspect, the antibodies are conveniently obtained from hybridomas prepared by immunizing an animal with the observed cleavage fragments or cleavage site specific analogueue peptides derived therefrom. Thus, the antibodies can be obtained by immunizing animals or screening antibody libraries with antibody cleavage fragments including F(ab′)₂ and scIgG, or N-terminal truncations or structural analogues thereof In one embodiment, the peptides used for generating the antibodies are selected from the 14-mer peptides fragments of IgG1 shown in SEQ ID NO: 5-11, where the C-terminal residue of the polypeptide or peptide represents the residue upstream (N-terminal side) of the cleavage site as shown in Table 1 of the residue cleavage pairs. Fragments comprising the hinge motif, e.g.—T-C-P-P-C- of IgG1 (residues 7-11 of SEQ ID NO: 1), will be multimeric due to disulfide bond formation, unless the cysteine residues (C) have been replaced with e.g. alanine (A) or serine (S) residues thereby creating a form of chemical homolog of the cleavage peptide.

In a specific embodiment, the antibody is generated using an 8-mer peptide corresponding to the sequence of amino acids on the amino terminal side of the MMP-3 cleavage site (TCPPCPAP, residues 7-14 of SEQ ID NO: 1), or extended peptides corresponding to the glutamyl endopeptidase site (TCPPCPAPE, residues 7-15 of SEQ ID NO: 1); or the IdeS site (TCPPCPAPELLG, residues 7-18 of SEQ ID NO: 1). When used as immunogens, the peptides can conveniently be covalently attached to keyhole limpet hemocyanin (KLH) or another carrier protein, such as serum albumin, via the N-terminus or through an added linker residue or peptide.

Antibodies with the desired specificity can thus be obtained using any of the hybridoma techniques well known in the art, see, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2^{nd} Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001), each entirely incorporated herein by reference. An antibody of the invention can include or be derived from any mammal, such as but not limited to a human, a mouse, a rabbit, a rat, a rodent, a primate, or any combination thereof and includes isolated human, primate, rodent, mammalian, chimeric, humanized and/or CDR-grafted anti-integrin antibodies, immunoglobulins, cleavage products and other specified portions and variants thereof.

Phage-displayed antibody or peptide libraries may also be used to identify novel binding domains with the desired specificity to scIgG and other antibody fragments.

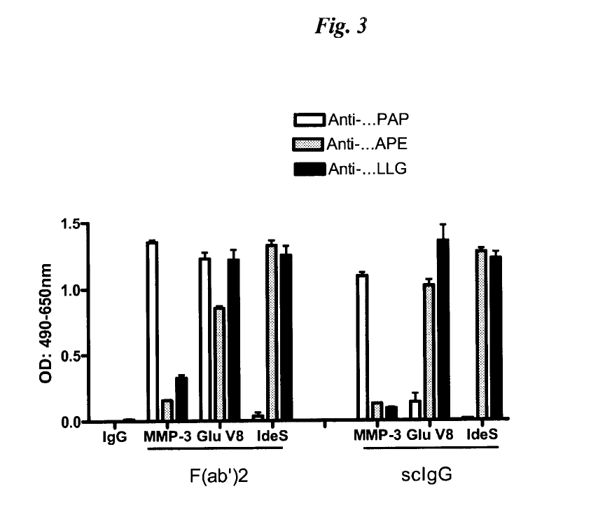

In raising or selecting antibodies or other binders useful in the present invention, the specific reagents used for this purpose are a further aspect of the invention. The specific immunogens or test reagents developed for this purpose are characterized as comprising residues around the hinge core of the IgG1, including but not limited to the residues SCDKTHTCPP CPAPELLGGP SVFLFP (SEQ ID NO: 1) as shown in FIG. 3. Hinge regions of other human isotype antibodies that produce antibody fragments upon contact with proteolytic enzymes may also serve as sources of analogue for the purposes of creating, selecting or testing antibodies or other binding molecules to enzymatic cleavage products. An analogueous region of the human IgG4 heavy chain includes residues TCNVDHKPSN TKVDKRVESK YGPPCPSCPA PEFLGGPSVF LF (SEQ ID NO: 2) and for IgG2 and IgG3 as shown in SEQ ID NOS: 3 and 4, respectively. In each case, the peptides consist of at least 5 contiguous amino acids selected from the human IgG hinge region sequences of SEQ ID NO: 1, 2, 3, or 4 that are on the amino terminal side of a protease cleavage site. In one aspect, the specific immunogen or peptide used for generating the antibodies comprise at least the hinge core of the IgG1, defined as the residues -C-P-P-C-. In a specific embodiment, the peptide is a 12-mer peptide analogue of the human IgG1 lower hinge and adjoining CH2 domain having the sequence TCPPCPAPELLG (residues 7-18 of SEQ ID NO: 1).

A general method for creating peptide fragments useful in generating, selecting or testing antibodies or other binding molecules to proteolytic cleavage products is to a) identify the N-terminal residue of a pair of residues of an antibody heavy chain cleaved by a protease such as those exemplified by specific proteases in as shown in Table 1, b) define from 5-14 or more upstream residues from that cleavage site where the N-terminal residue will become the C-terminus of the defined sequence and c) produce the peptide in sufficient amounts for the desired purpose(s). Optionally, any cysteine residues can be substituted with serine or alanine or other amino acid where no reactive side groups are present or reactive side group has been irreversible or reversible blocked. Peptides such as those described are those selected from SEQ ID NO: 5-11 or N-terminal truncations thereof. The peptides may be labeled, conjugated or cross-linked or used in admixture one with another or with adjuvants for the purposes of testing binding or as immunogens or panning targets for use e.g. in selecting binders from a phage display library.

The present invention further provides, in one aspect, isolated nucleic acid molecules comprising, complementary, or hybridizing to, a polynucleotide encoding the aforementioned specific peptides or antibodies thereto, comprising at least one specified sequence, domain, portion or variant thereof The present invention encompasses isolated nucleic acids encoding at least one isolated monoclonal antibody having specificity for the scIgG as described herein and a nucleic acid vector comprising the isolated nucleic acid, and/or a prokaryotic or eukaryotic host cell comprising the isolated nucleic acid. The host cell can optionally be at least one selected from E. Coli, COS-1, COS-7, HEK293, BHK21, CHO, BSC-1, Hep G2, 653, SP2/0, 293, HeLa, myeloma, lymphoma, yeast, insect or plant cells, or any derivative, immortalized or transformed cell thereof. Also provided is a method for producing at least one antibody of the invention, comprising translating the antibody encoding nucleic acid under conditions in vitro, in vivo or in situ, such that the peptide or antibody is expressed in detectable or recoverable amounts.

### Methods of Use

### Diagnostics

An antibody prepared using IgG cleavage peptides can be used as detection reagents useful in indentifying, or confirming disease pathology or processes resulting from, causing, or is otherwise associated with proteolytic activity and proteolytic enzymes (proteases). Such diseases and processes include those precipitating or aggravating, produced by, or resulting from infection, stroke, vascular disease, myocardial infarction and several other acute and chronic inflammatory disorders. Applicants have demonstrated that one particularly useful biomarker of the proteolytic activity is scIgG, which is detected at increased levels in some of the aforementioned disorders. As scIgG is generated locally at the site of the pathology or pathological process or infection, scIgG provides a unique and specific marker of such processes as a gauge of the involvement of specific tissues or cell types at the disease site.

In one embodiment of the method of the invention, a sample is obtained from a subject suspected of having, having had, or having been treated for a disease characterized by elevated levels of proteases. The sample is contacted with a binding agent, such as an antibody preparation, having specificity for IgG cleavage fragments known to result from contact between the disease stimulated protease and a population of serum IgG.

The method of the invention can be used to assess whether patients previously diagnosed with a disease or condition are at risk for advanced disease (e.g. cancer metastases, aggressive tumor growth, persistent infection, etc.).

In some cases, for example in the cancer patient, the detection of scIgG may be useful to indicate advanced disease progression involving metastatic spread which is known to involve elaboration of proteolytic enzymes, especially MMPs. In some aspects, neoplastic disease shares these mechanisms generally with inflammatory processes, tissue repair, and healing (Coussens, L. M. and Werb, Z. 2002. Nature 420 (19): 860-867). Other studies have shown that, for example, lipid lowering correlates both with a reduction in the risk for cardiac and vascular events, e.g. thrombosis, and with a reduction in MMPs such as MMP-2 and MMP-9 and that these enzymes are produced by atherosclerotic plaques (Deguchi, J, Maanori, A., Ching-Hsuan, T. et al. 2006 Circulation 114: 555-62). Thus, the methods of the invention are particularly applicable but not limited to patients with severe arthritic syndromes (RA, ankylosing spondylitis), certain cancers (especially inflammatory breast cancer), severe coronary arterial settings (myocardial infarction and congestive heart failure) and other diseases like asthma. The method of the invention may be used to distinguish those diseases and conditions in which the pathophysiology involves or induces protease capable of acting upon IgG from other pathologies not characterized by enhanced elevated levels of secreted proteases or wherein the proteases do not cleave IgG.

Thus, while the method of using the reagents described herein are specific for detection of the cleaved fragments, more specific analysis of the cleaved fragments could include an analysis of the binding specificity of the variable regions of the cleaved antibody. For example, a solid phase assay which combines antigen binding selectivity with fragmented antibody detection could be used to determine whether certain antigens and proteases are co-localized in a subject thereby providing information about the nature of the tissue, disease, or pathology at the site of proteolytic activity.

Drawing blood is the most frequently practiced form of tissue sampling from subjects, human or animal, healthy or ill. In so far as scIgG is found systemically, and is not restricted to the site of formation, that is, the site of the protease activity, it is a reporter marker for disease activity which may localized in specific compartments. One such compartment is the synovial fluid. Thus, blood or serum collection provides a convenient and feasible source for detection of early disease using the reagents and methods provided by the present invention. Alternatively, sampling of local settings like RA synovial fluid, lung exudates, biopsies, and the like could also be applied to patients at any stage including diagnosis or in patients with advanced disease. Cleaved antibody fragments may be detected in such tissue samples by direct staining (immunohistochemical methods) or in fractioned samples derived from the samples.

Tissue samples, including blood, should be treated so as to inhibit any residual active proteases. Chelation of metals (e.g. EDTA) effectively inhibits MMPs. Iodoacetamide blocks cysteine proteases (e.g. IdeS), serine proteases can be blocked with DFP and similar compounds. Active proteases are present in synovial fluid and should be processed accordingly. Samples may also be maintained frozen until the time of assay. Once the samples have been appropriately processed, the scIgG specific reagents of the invention may be used in any antibody-based techniques such as ELISA, bead-based formats, RIAs, known to those skilled in the art or yet to be developed.

The anti-IgG proteolytic cleavage fragment reagents of the invention may be packaged in a kit for research or diagnostic use and for commercial sale along with other reagents such as buffers and standards such as intact human IgG and known quantitities of cleaved IgG along with instructions for the measurement and, if desired, quantitation of IgG proteolytic cleavage fragments in tissue samples harvest from subjects.

### Antibody Therapy

Antibodies of the invention immunospecific for hinge peptide cleavage fragments are capable of binding the remnants of proteolytically cleaved IgG which retain antigen binding domains, e.g. Fab, F(ab′)₂, scIgG , and thus restore the Fc-related binding characteristics and attendant effector functions by providing an intact Fc-region. Thus, the antibodies created by the methods taught herein or having the property of binding to proteolytically created antibody fragments in vivo may be useful as therapeutic molecules. The anti-IgG cleavage fragment antibodies of the present invention can be used to treat patients in which a disease characterized by disease induced proteolytic cleavage of IgG. In one aspect, the anti-IgG cleavage fragment antibodies may be used to restore effector functions to antibody fragments which retain target specific binding capability.

The anti-IgG cleavage site antibody intended for therapeutic or prophylactic treatment of human disease or pathology may be prepared by the methods described herein above using the peptides of the invention as immunogens or selection reagents. Other binding domains specific for the cleaved hinge fragment may also be used so long as the binding domain is associated with an Fc-domain capable of restoring the effector functions, such as CDC or ADCC, to an immunospecific IgG cleavage product. Of course, modified or variant Fc sequences are also encompassed by the invention for the purposes of enhancing some FcR-driven interactions and attenuating others. Modified Fc regions are taught in, e.g. U.S. Pat. No. 6,737,056, U.S. Pat. No. 7,083,784, U.S. Pat. No. 7,317,091, U.S. Pat. No. 7,355,008, U.S. Pat. No. 7,364,731, U.S. Pat. No. 7,371,826, U.S. Pat. No. 7,632,497, U.S. Pat. No. 7,670,600, US20040002587A1, WO06105338A2, WO200905849, and WO2009086320.

Thus, the invention are provided including nucleic acid sequences, vectors, and host cells for the recombinant production of anti-IgG cleavage product antibodies or Fc-fusion proteins capable of binding a protease cleaved IgG and restoring effector functions.

### Vaccination

Antibodies of the invention immunospecific for cleavage site specific fragments, capable of binding the remnants of enzymatically cleaved IgG which retain antigen binding domains, e.g. Fab, F(ab′)₂, scIgG , and thus restoring the Fc-related binding characteristics and attendant effector functions of the antibody by providing a functional Fc-region, may be induced in a subject by immunization with a cleavage fragment peptide as disclosed herein. The anti-IgG cleavage product antibodies can be prepared by immunizing a host animal with a protease cleavage site specific peptide or proteolytically cleaved IgG fragments, and recovering the antibodies from the animals' serum. In such a method, the immunized animal is a source of the antibodies of the invention from which an antibody to be used as a reagent for a diagnostic test or, alternatively, to be used therapeutically, is prepared by methods described or known in the art. In a particular embodiment, a human subject is immunized with a protease cleavage site specific peptide or proteolytically cleaved IgG fragments and the anti-IgG cleavage product is generated in vivo. In one embodiment, the protease cleavage site specific peptide immunogen is selected from the group consisting of at least five (5) contiguous amino acids selected from the human IgG hinge region sequences of SEQ ID NO: 1, 2, 3, or 4 that are on the amino terminal side of a protease cleavage site such as the sequences of SEQ ID NOs. 5-11, N-terminal truncations and chemical homologs thereof.

The use of an cleavage site specific immunization or “vaccination strategy” will provide universal restoration of the inherent Fc-domain functions of IgGs independent of the origin of the protease (i.e. of bacterial origin or host origin in the case of proteolytically-enriched tumors) limited only to the specificity of the cleavage site specific antibody for the epitope formed by the residue specific cleavage within the IgG. An individual may be vaccinated with more than one cleavage site peptide or analogueue peptide for a broader spectrum of antibody restorative capabilities, if so desired. Alternatively, a patient may be treated with a cleavage site specific antibody before, during or after being vaccinated to produce an cleavage site specific antibody response. In a particular embodiment, the individual is vaccinated with a disease specific cleavage site specific peptide or an analogueue peptide before, concurrent with, or following the administration of a targeted antibody preparation designed to treat a given disease state where the targeted antibody activity involves effector function, and the antibody is subject to cleavage by one or more IgG cleaving proteases. In this manner, the effector function of the targeted antibody can be restored and the effect of the antibody treatment is enhanced or restored while the targeting function of the antibody has not been altered. In one embodiment, the disease being treated in a subject is characterized by elaboration of one or more IgG-cleaving proteases (see Table 1).

Methods of vaccination are well known in the art and, in particular, it is known that small antigens, or haptens, and linear peptides are more immunogenic when a plurality thereof are conjugated to a carrier molecule which may also be immunogenic such as keyhole limpet hemacyanin (KLH). Numerous conjugation methods are known in the art and are described, for example, by G. T. Hermanson in “Bioconjugate Techniques”, Academic Press, 1996. Briefly, conjugations of a hapten to a carrier is generally effected by means of linkers or, more appropriately cross-linkers, which consist of linear molecules of various length bearing reactive functional groups at both ends. In homobifunctional linkers (i.e. glutaraldehyde) the two functional groups are identical: in heterobifunctional linkers, they are different. The detailed conjugation chemistries are well known. As the targeted IgG cleavage fragments retain the antigen specific binding domains associated with the N-terminus portions of the immunoglobulins (variable region, CH1 and, in some case, some of the core hinge domain) the antibody response should be directed to the portion of the analogueue peptide representing a new C-terminus at the cleavage site. Therefore, it is desirable to conjugate the N-terminus of the analogueue peptide and present a free C-terminal residue as the immunogen.

In a vaccination method, adjuvants (for example, aluminum-containing adjuvant (Alum), Incomplete Freund's Adjuvant (IFA), Complete Freund's Adjuvant (CFA)) can be used with the unconjugated or conjugated hinge region analogueue peptides to direct the immune response to produce antibodies (humoral or Th2-driven) and direct the binding domains against the cleavage site specific fragments. CD40 agonists such as the natural ligand gp39 can be used to stimulate the immune response. A CD40 agonist that activates a CD40 receptor preferably on an antigen presenting cell, preferably a dendritic cell is one example.

### HumanVaccines

Human vaccine preparations have been developed which are safe and effective. To enhance the immunogenicity of recombinant protein-based vaccines, adjuvants are required. The most widely used adjuvants are insoluble aluminium salts, generically called alum, such as boehmite and aluminium hydroxyphosphate. Alum adjuvants induce predominantly a Th2-type cytokine response (Lindblad, 2004 ; Raz & Spiegelberg, 1999 ; Valensi et al., 1994). Therefore, alternative adjuvants may be required for the successful development of a peptide vaccine.

Polymeric microparticle encapsulation of antigens have been evaluated as vaccine adjuvants (Eyles et al., 2003J Drug Target 11, 509-514; Singh et al., 2004 Expert Opin Biol Ther 4, 483-491). Microparticles formed of poly(DL-lactide co-glycolide) (PLG) are well known in the art.

Various oil-in-water emulsions have also been developed as alternative adjuvants to alum. The most advanced of these is a squalene oil-in-water emulsion (MF59), which is a potent adjuvant with an acceptable safety profile. The influenza vaccine product called Fluad comprises MF59. Like alum, MF59 can promote antigen uptake by dendritic cells in vivo. Moreover, it has been shown that, after i.m. injection, MF59 is internalized by APCs that migrate to the lymph node. Besides promoting antigen delivery, MF59 might also act as a local pro-inflammatory adjuvant as it was observed to promote an influx of blood mononuclear cells after i.m. injection.

Other molecules that can be used in combination with alum, MF59, or microparticles include, but are not limited to; CpG to enhance Thl-type responses to vaccines, GM-CSF, and IL-2.

The prepared immunogen with or without admixed adjuvants or “vaccine” may be administered to the subject by any suitable route, such as but not limited to intradermally, subcutaneously, intranasally, and intramuscularly.

Various protocols may be used for vaccination. One protocol contemplated as one aspect of the invention is the prime-boost protocol wherein a first vaccine composition that comprises the desired immunogen, a prime vaccine composition, is administered in conjunction with a boost vaccine composition that comprises a corresponding immunogen that differs in form from the immunogen of the prime vaccine composition. The boost vaccine composition may be administered at the same time as the prime vaccine composition or it may be administered at some time following the initial administration of the prime vaccine composition. The prime and boost vaccine compositions may be administered via the same route or they may be administered via different routes. If the prime and boost vaccine compositions are administered at the same time they may be administered as part of the same formulation or as different formulations. Both the prime vaccine composition and the boost vaccine composition may be administered one or several times. Thus some doses of the prime vaccine may be administered after the administration of a dose of the boost vaccine. It is within the skill of one with ordinary skill in the art to optimize the vaccination protocol using these and other known or yet to be discovered variation of routes of vaccine administration and timing for vaccine administrations.

While having described the invention in general terms, the embodiments of the invention will be further disclosed in the following examples.

### EXAMPLE 1

### Use of Peptide Immunogens to Elicit an Immunoglobulin Cleavage Site Specific Antibody Preparation

The determination of the presence of host (patient) antibody fragments produced by endogenous proteases requires a reagent which selectively binds to the cleaved IgG but not intact IgG. Both identification of the cleaved component and a quantitative difference between fragment content in samples from patients with disease as compared to the normal population should be able to be assessed using the reagent.

The detection of unknown, but likely small amounts of IgG fragments in solutions containing relatively high concentrations of intact IgG is difficult. Although scIgG has been noted as a possible IgG cleavage fragment (Gearing 2002 supra), quantitation in human samples has not been previously performed. For this purpose, a reagents with the necessary specificity were generated in rabbits having with a high degree of specificity for cleaved but not intact IgG.

Three conjugated, and progressively longer single-chain peptide analogues of the human IgG1 hinge were used for immunization (at Invitrogen Corporation). An 8-mer peptide corresponding to the sequence of amino acids on the amino terminal side of the MMP-3 cleavage site was covalently attached to keyhole limpet hemocyanin (KLH) via the N-terminus (TCPPCPAP, residues 7-14 of SEQ ID NO: 1). Extended peptides corresponding to the glutamyl endopeptidase site (TCPPCPAPE, residues 7-15 of SEQ ID NO: 1) and the IdeS site (TCPPCPAPELLG, residues 7-18 of SEQ ID NO: 1) were separately prepared as immunogens. New Zealand rabbits (two per immunogen) were immunized by subcutaneous injection of 0.2 mg conjugated peptide in complete Freund's adjuvant and re-boosted three additional times with 0.1 mg antigen in incomplete Freund's adjuvant on days 14, 42 and 56. Serum was collected at 4, 8 and 10 weeks and pooled per immunogen for antibody purification. The immune titers were monitored by an ELISA based on solid phase antigen peptide.

Affinity purification of antibodies employed the respective peptide antigens immobilized on an activated support. The antiserum from the two rabbits immunized with the same antigen was pooled and passed through the antigen column after which the column was extensively washed. Specific antibodies were eluted as low affinity and high affinity pools using 3M KSCN and 0.1M glycine, pH 2.5, respectively. The two pools yielded indistinguishable binding characteristics and were used interchangeably and/or pooled. The three separately eluted pools of bound antibodies were next subjected to a second affinity adsorption step, this time on a column containing an intact antibody comprising human IgG1 heavy chain constant regions (Mab3). The intent of the second affinity chromatography step was to remove undesirable antibodies that might recognize intact IgG. However, little or no rabbit antibody was adsorbed to the IgG column suggesting that the population of antibodies was reactive only with the “cleaved” sequence with its exposed carboxy terminus.

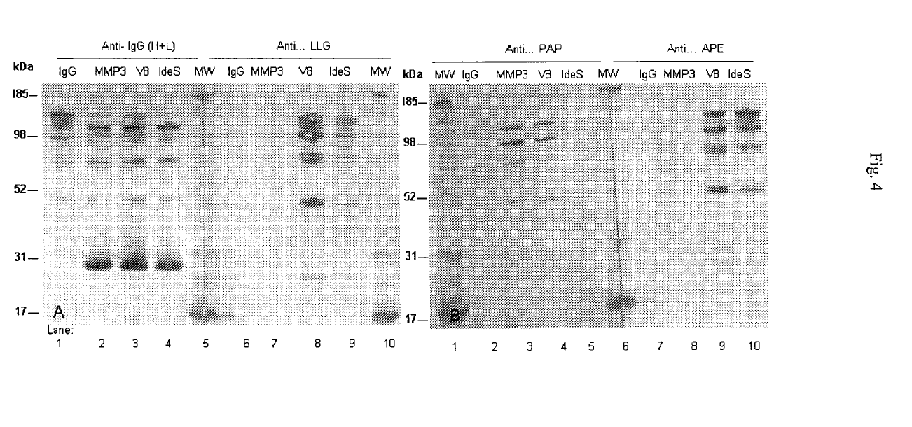

The individual affinity-purified rabbit anti-peptide antibodies were tested for their ability to bind to enzymatically-generated fragments of human IgG as well as intact IgG by ELISA (FIG. 3). The purified antibodies from the rabbits immunized with KLH conjugated to residues 7-14 of SEQ ID NO: 1 (the MMP-3 site analogue) did not bind intact IgG and were highly specific for scIgG and F(ab′)₂ produced by digestion of IgG with MMP-3. This antibody preparation showed minimal reactivity to scIgG and F(ab′)₂ produced with V8 protease or IdeS. In contrast, the antibodies obtained from rabbits immunized with the V8-cleavage site hinge peptide analogue (residues 7-15 of SEQ ID NO: 1) and the IdeS-cleavage site hinge peptide analogue (residues 7-18 of SEQ ID NO: 1) showed a cross-reactive binding profile for scIgGs and F(ab′)₂ produced by either of these two enzymes. However, these preparations showed minimal reactivity for the MMP-3 digested products. None of the antibody preparations bound to intact IgG and none of the antibody preparations was comparably reactive with fragments, including F(ab′)₂ and scIgG, produced by three different enzymes as shown in FIG. 4.

The intended use of the anti-hinge reagent is the detection of scIgGs and F(ab′)₂ (and other potential fragments) that are produced in complex in vivo settings by enzymes present in disease specific tissues or produced by disease specific cell types or cell populations, e.g. infiltrating macrophages or neutrophils. For optimal coverage of potential IgG fragments, it was considered preferable to have as broad a profile of cleavage site recognition as possible. For this reason, a mixture of each of the three rabbit antibody pools was prepared at 0.33 mg/mL of each component (total=1 mg/mL) for use in subsequent Western blotting and serum-based ELISA tests. This pooled reagent is referred to as RAH-1.

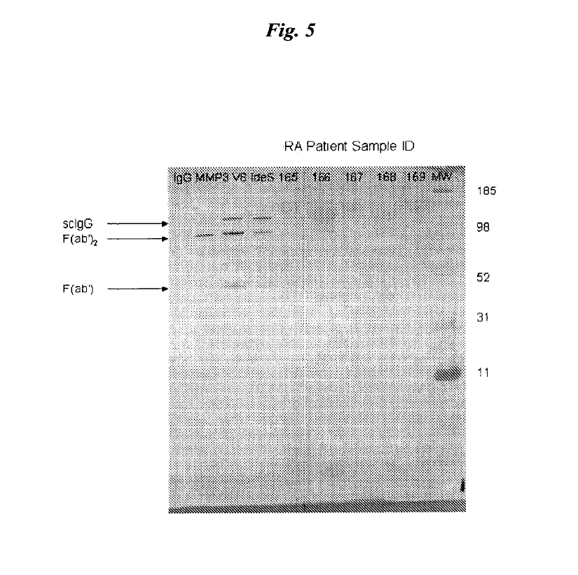

The RAH-1 reagent was tested for its ability to detect IgG fragments in another inflammatory fluid, the synovial fluid of a patient with rheumatoid arthritis (RA). Synovial fluid samples from RA patients were purchased commercially As shown in FIG. 5, the RAH-1 preparation did not react with the intact IgG , but detected scIgG, F(ab′)₂ possibly Fab′ from all 3 protease digests. For all five synovial fluid samples from RA patients, a band was detected at the approximate size of scIgG, F(ab′)₂ and Fab′, suggesting that these proteolytic fragments were present within the synovial fluid from individuals with RA.

Serum samples from 10 subjects with rheumatoid arthritis (RA) and from 10 age- and gender-matched healthy individuals were obtained from Genomics Collaborative. The ELISA results indicated that 4 of the 10 subjects with RA demonstrated serum scIgG concentrations >60 μg/mL. In the healthy control group, scIgG concentrations ranged from <8.2 μg/mL to 52.7 μg/mL. The samples for this comparison were not rigorously selected for stage of disease, treatment regimens, etc., however, the present assays on these commercial samples suggest that elevated scIgG concentrations can be detected in patients with disease.

In another test, a commercial source of serum from diseased individuals was identified as Genomics Collaborative (now SeraCare Life Sciences Inc.). Small volumes (300 microL) of serum from 10 different individuals within each of eight diseases were purchased. The disease categories were rheumatoid arthritis, osteoarthritis, asthma, type-1 diabetes, breast cancer, lung cancer, myocardial infarction, and congestive heart failure. In addition, serum from 28 age-matched and gender-matched normal healthy volunteers were obtained from this vendor as controls.

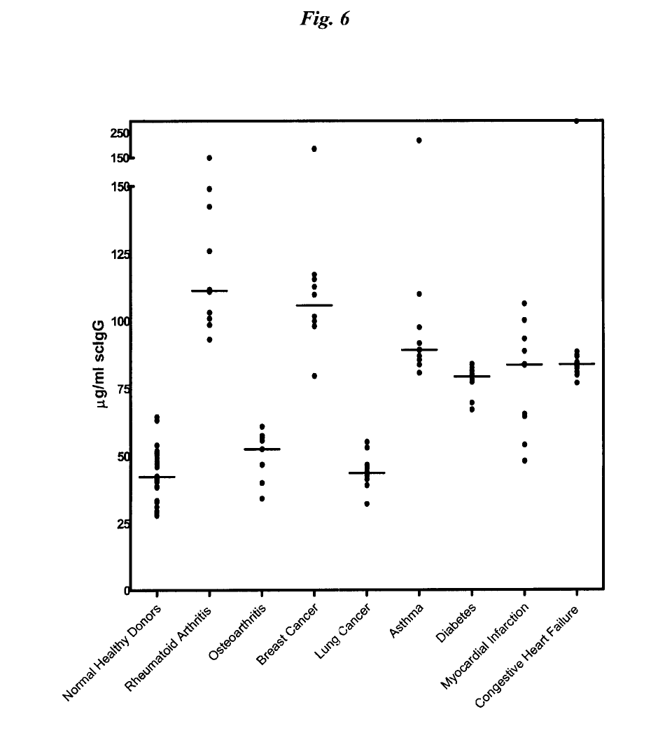

Using an ELISA format assay, the samples were analyzed and the results shown in FIG. 6. The assay, based on the selectivity of the RAH-1 reagent, demonstrated that IgG cleavage products comparable to those generated by known specific proteases are clearly detectable and above levels maintained in healthy or normal donors for an inflammatory autoimmune disease, rheumatoid arthritis. In contrast, patients with osteoarthritis showed levels which were similar and in the range of those for the normal individual's samples.

### EXAMPLE 2

### Preparation of Anti-Hinge IGG Monoclonal Antibody

It would be desirable to produce a defined molecule, such as a monoclonal antibody, for manufacture and potential use in human patients which binds cleaved IgG and not intact IgG. The following procedure represents a method for generation of such a molecule.

A 12-mer peptide analogueue of the human IgG₁ lower hinge region and adjoining CH2 domain was the immunogen: TCPPCPAPELLG (residues 7-18 of SEQ ID NO: 1) which is a peptide analogueue of the IdeS cleavage site of human IgG1. The naturally occurring cysteines were replaced by alanines to give the variant TAPPAPAPELLG (SEQ ID NO: 12). An N-terminal cysteine was added to allow for conjugation to keyhole limpet hemocyanin (KLH) by standard chemical methods for reaction to free sulhydryls.

New Zealand white rabbits (3) were immunized with 0.5 mg KLH peptide in complete Freund's adjuvant using multiple subcutaneous sites (5). The animals were boosted with the 0.25 mg immunogen in incomplete Freund's adjuvant at three-week intervals for a total of 4 additional immunizations.

The serum antibody titers to a BSA-conjugated version of the same peptide were monitored during the course of the immunization by standard ELISA methods. Animals (2) were chosen for splenectomy based on the titer data. Rabbit hybridomas were generated from spleen-derived lymphocytes fused with a rabbit fusion partner cells (Spieker-Polet, 1995 PNAS USA 92(20):9348-9352). Cell growth was examined 2-3 weeks after fusion in multiple plates.

Positive hybridomas were screened via ELISA on plates coated with the BSA-immunogen peptide conjugate. Multiple positive clones from each fusion were identified. Further screening involved binding to intact IgG1 and various enzymatically-generated F(ab′)2 fragments of IgG1. From these screening and counter-screening tactics, three clones (designated 33-2, 91-2, and 68-6) were chosen based on strong selectivity of binding to the immunogen peptide and to F(ab′)2 fragments with C-termini at or near the C-terminus of the immunogen peptide and with minimal binding to intact IgG1. The positive hybridomas were subcloned and expanded.

Rabbit IgG was purified from individual cell supernatants by standard methods including chromatography on immobilized protein A. The specificity of the purified rabbit IgGs for binding to peptide analogues of the human IgG1 hinge region, as well as intact IgG and purified IgG fragments created of single or doubly cleaved (F(ab′)2) cleaved with mAbs using IdeS and MMP-3 enzymes were tested in standard ELISA protocols. Briefly, the peptides which were synthesized by standard peptide chemistry and were N-terminally biotinylated were captured on streptavidin-coated wells. The IgG and fragments were directly coated at 10 μg/mL. Binding of rabbit mAbs was detected by well-characterized goat anti-rabbit IgG Fc-horseradish peroxidase and OPD substrate systems.

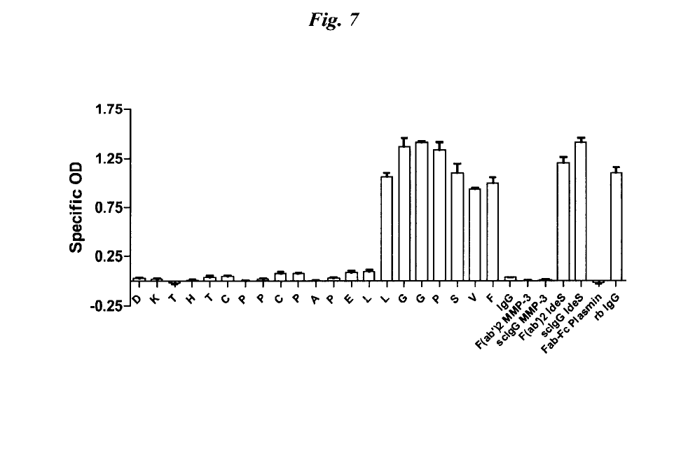

The ELISA results for rabbit mAb 91-2 are shown in FIG. 7. There was a clear selectivity of binding for lower hinge peptides terminating at any of the residues 16-23 of SEQ ID NO: 1 (L-L-G-G-P-S-V-F). There is little or no binding to the peptides terminating at any of the upstream residues corresponding to those segments of the upper hinge, core hinge or early lower hinge encompassed by 3-15 of SEQ ID NO: 1 (D-K-T-H-T-C-P-P-C-P-A-P-E). There was negligible binding to the MMP-3 generated F(ab′)2 fragment and scIgG fragment (in agreement with the lack of binding to the peptide analogues of the MMP-3 cleavage site between residues 14 and 15 of SEQ ID NO: 1 (ending in P-A-P). In contrast, there was substantial binding to the Ides-generated F(ab′)2 fragment and scIgG which should have the C-terminal sequence (-P-A-P-E-L-L-G). Thus, the rabbit mAb binding specificity conformed well to the immunogen to which it was elicited. Directly coated rb (rabbit) IgG was a positive control.

### Complement Assay

WIL2-S cells, a lymphoblastoid B-cell line expressing CD20 (ATCC CRL-8885), were used as target cells for CDC assays. 50 μl of cells were added to the wells of 96-well plates for a final concentration of 8×10⁴ cells per well in RPMI, 5% heat-inactivated FBS, 0.1 mM nonessential amino acids, 1 mM sodium pyruvate, penicillin (500 U/ml), streptomycin (500 U/ml), 2 mM L-glutamine. An additional 50 μl was added to the wells with or without antibodies of various concentrations and the plates were incubated at room temperature for 2 hours. 50 μl of 10% rabbit complement (Invitrogen) was added to the wells and the plates were incubated for 20 minutes at 37° C. All samples were performed in triplicate. The plates were centrifuged at 200 g for 3 minutes and 50 μl of supernatant was removed to separate plates and CDC was measured with lactate dehydrogenase (LDH) cytotoxicity detection kit (Roche). Absorbance was measured using a Spectra max Plus 384 (PerkinElmer). Data were normalized to maximal cytotoxicity with Triton X-100 (Sigma Aldrich) and minimal control containing only cells and complement alone.

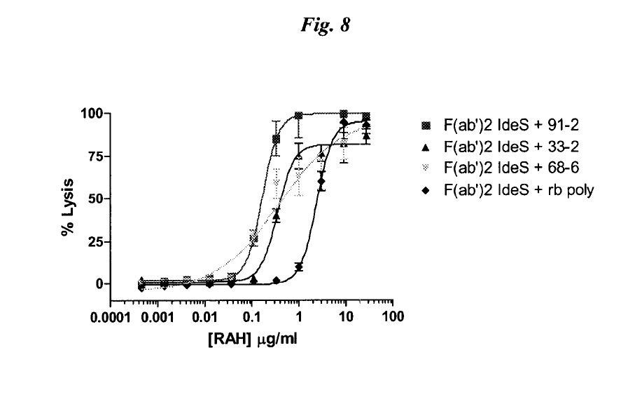

FIG. 8 shows that the 3 rabbit cleavage site specific mAbs were able to restore complement dependent cell lysis to the target cells when titrated in the presence of a fixed concentration of the F(ab′)2 fragment of an antibody that binds CD20. The rabbit mAbs were more effective, and, at lower concentrations than a polyclonal rabbit cleavage site specific mAb preparation (a component of the same detection system for serum scIgG described earlier). Intact antibody to CD20 was active, as expected, but its F(ab′)2 fragment and scIgG version were not active alone. The rabbit cleavage site specific mAbs were not able to direct cell lysis in the absence of cell-binding F(ab′)2 fragment. These results establish that monoclonal cleavage site specific antibodies can reconstitute complement-mediated effector function to otherwise inactive proteolytic cleavage products of IgG1.

In order to test the restoration of effector functions in human system, the rabbit antibody variable domains of Mab 91-2 were cloned, fused to human constant domains and expressed in HEK293 cells. The resulting rabbit-chimeric Mab was designated 2095-2. The antibody specificity was again tested by binding to cleavage site peptides in an ELISA format and confirmed that the highest affinity was for the peptide terminating with -P-A-P-E-L-L-G.

The specificity of binding was further examined using a Fab fragment of the 2095-2 antibody using a surface plasmon resonance platform (Biacore). Briefly, the Fab was immobilized to a CM5 sensor chip in a Biacore 2000 at three different surface densities (8500, 1650, and 350 RU) using standard NHS/EDC coupling. The running buffer contained 10 mM HEPES pH 7.4, 150 mM NaCl, and 0.01% Tween-20. Data were collected at 25° C.

Three N-terminally biotinylated peptides were tested for binding of to the immobilized Fab: WT, representing the expected sequence of a peptide fragment from a human IgG1 cleavage site after cleavage by the Streptococcus pyogenes IdeS protease; E233A, representing a variant of having a single alanine substitution at the amino acid residue four positions upstream of the C-terminus; and L234A, a variant having a single alanine substitution at the amino acid residue three positions upstream from the C-terminus. The peptides were modified by substituting serine for cysteine to make them compatible with the coupling chemistry and preserve the monomeric structure.

Peptides Wild type and E233A were tested in a 3-fold dilution series using 588 nM and 2.9 uM as the highest concentration, respectively. Peptide L234A was tested at 14.7 uM as the highest concentration in a 2-fold dilution series. Each peptide concentration series was tested in triplicate over the three different density Fab surfaces. The association and dissociation were monitored for 1 minute. Surfaces were regenerated with a 12 second injection of 1/500 dilution of phosphoric acid. The response data from each surface were globally fit to determine the binding constants summarized in the table below. Each peptide interaction fit very well to a simple 1:1 (Langmuir) model.

The association rates (kₐ in M-1s-1)) varied by less than a factor of 10, however, the dissociation rates (k_{d} in s-1) varied by more than 200-fold. The calculated K_{D} in nM for the three peptides is shown below (Table 2).

TABLE 2

| | Peptide | kₐ(M-1s-1) × 10⁻⁵ | k_{d} (s-1) | K_{D} |
|---|---|---|---|---|
| Wt | Biotin-TSPPSPAPELLG | 5.22 ± 0.04 | 0.00443 ± 0.02 | 8.5 nM |
| | (SEQ ID NO: 13) | | | |
| E233A | Biotin-TSPPSPAPALLG | 7.29 ± 0.03 | 0.1070 ± 0.04 | 146.8 nM |
| | (SEQ ID NO: 14) | | | |
| L234A | Biotin-TSPPSPAPEALG | 1.24 ± 0.01 | 1.03 ± 0.01 | 8340 nM |
| | (SEQ ID NO: 15) | | | |

These results indicate that the monoclonal antibody 2095-2 is highly specific for the binding to the immunogen used which is an analogueue of the IdeS cleavage site peptide from the N-terminal (upstream) sequence of human IgG1 and is in monomeric conformation due to the removal of cysteine residues.

### EXAMPLE 3

### In Vivo Model for Cleavage Site Specific Antibody

In order to test the capacity of cleavage site specific antibodies to rescue cleaved IgGs in vivo, a model for bacterial infection employing a “tissue cage” (Fernandez J A, et. al. (1997) Antimicrob. Agents Chemother. 43(3):667-671) was adopted. In this system, a wiffle ball is surgically implanted subcutaneously in the dorsal cervical area. The model allows the infectious agents to be localized within an easily accessible fluid compartment, a tissue pouch, and has been used to assess the efficacy of antibiotics against bacteria.

In the present system, the rabbit wiffle fluid ball was found to contain approximately 1.5 mg/ml IgGs, in contrast to 5-10 mg/ml of IgGs reported in rabbit serum. Our hypothesis is that infection of rabbits with the GluV8-expressing bacteria S. aureus would result in cleavage of the rabbit IgGs within the wiffle ball. Furthermore, vaccination of rabbits with a GluV8-cleaved IgG cleavage site specific peptide analogueue would result in robust cleavage site specific titers that would provide a measure of protection against S. aureus.

In order for the model to provide a positive result in demonstrating that rabbit cleavage site specific antibodies can provide some level of protection against S. aureus infection, four factors must be operating. First, GluV8 must be capable of cleaving rabbit IgGs. Second, rabbits must have either some pre-existing immune reactivity to S. aureus antigens or they must be able to generate an antibody-mediated immune response against S. aureus shortly after infection as these will become the subject IgG. Third, the vaccination approach must yield cleavage site specific antibodies of sufficient affinity and specificity to bind the cleaved rabbit IgGs. Finally, animals immunized with the cleaved antibody analogueues must demonstrate a measurably different response than non-immunized animals.

We previously showed that GluV8 cleaves human IgG1 between the amino acids E233 and L234 (WO2009/023457A1, Ryan M H, et al. (2008) Mol Immunol 45(7):1837-1846) located in lower hinge region in vitro. The sequence of the rabbit IgG lower hinge region in this region is identical to the human IgG 1 sequence except for the single amino acid change, A231P. As the GluV8 cleavage site it was expected that the enzyme would act on rabbit IgG.

| | |
|---|---|
| Human IgG1 | ₂₂₉CPAPELLGG₂₃₆ |
| Rabbit IgG1 | ₂₂₉CPPPELLGG₂₃₆ |

Rabbit IgGs from the serum and from the S. aureus containing wiffle ball fluid of rabbits three days after inoculation were purified separately using a Protein A reagent. The resulting preparations of rabbit IgGs were analyzed by MALDI to determine the amount of IgG cleavage. The MALDI analysis of the rabbit serum IgGs revealed three primary peaks, corresponding to the singly-charged molecular ion of intact IgG (143,600 Da), the doubly-charged molecular ion of intact IgG (71,700 Da), and rabbit albumin (65,800 Da). In contrast, the MADLI analysis of wiffle ball rabbit IgGs contained the singly-charged molecular ion of an Fc fragment (52,800 Da), the doubly-charged molecular ion of an Fc fragment (25,500 Da), and a small peak corresponding to the singly-charged molecular ion of intact IgG (143,700 Da). These results indicated that rabbit IgGs purified from S. aureus containing wiffle ball fluid contained cleaved IgGs, whereas rabbit IgGs purified from the serum contained intact IgGs with no detectable IgG cleavage products. Therefore, we concluded that rabbit IgGs in vivo, like human IgG1 in vitro, are susceptible to cleavage within a microenvironment containing S. aureus.

With regard to the immune status of the study animals toward Staph. aureus, the presence of pre-existing immune reactivity in serum was tested by ELISA using two S. aureus-derived antigens. These were Staph. alpha toxin (alpha-hemolysin, Hla) and Staphylococcal Enterotoxin B (a toxin commonly associated with food poisoning). The pre-study serum from the six rabbits that subsequently were immunized with the hinge analogueue peptide, and the pre-innoculation serum from 6 non-immunized control rabbits were tested at 1:50, 1:200 and 1:800 dilutions for binding to the two toxins. A positive titer was quantified as the highest dilution at which optical density (490 nm) was two-fold or greater than the signal obtained in the corresponding control well (non-antigen coated). In addition, a human integrin and chicken egg albumin were tested as potential negative controls since it was unlikely that rabbits would have been exposed to such proteins. The results are tabulated below (Table 3).

TABLE 3

| | Incidence of positive | |
|---|---|---|
| Antigen | reactivity | Titers (n) |
| Staph alpha toxin | 8/12 | 1:50 (2), 1:200 |
| | | (1), >1:800 (5) |
| Staph enterotoxin B | 12/12 | 1:50 (3), 1:200 |
| | | (3), >1:800 (6) |
| Human integrin | 1/12 | 1:50 (1) |
| Chicken egg albumn | 4/12 | 1:50 (4) |
| (ovalbumin) | | |

### Results

The results indicated that there was a substantial incidence of anti-Staphylococcal toxin reactivity in the 12 study animals. In some rabbits, the titers would clearly have exceeded the maximum dilution tested (1:800). In contrast, the incidence of immune reactivity to the human and chicken antigens was markedly lower and was detectable only at the 1:50 dilution. These results supported the supposition that rabbits are naturally exposed to S. aureus in their lifetimes and should be capable of immediate targeting of S. antigens upon innoculation, or possess the ability to rapidly mount a recall response. These findings suggest that a spectrum of antibodies to Staph. aureus antigens may be present including antibodies to cell surface components.

### EXAMPLE 4

### Immunization

The procedure described in Example 3 verified that test subject rabbits have had previously developed immune titers to Staph. aureus antigens which would provide for natural bacterial targeting antibodies. To determine if rabbits would generated a sufficiently specific and robust cleavage site specific antibody response after vaccination, with cleavage site peptides, to protect against a bacterial challenge, the following experiment was perfomed where rabbits were immunized with a GluV8-cleavage site peptide analogueue (residues 7-15 of SEQ ID NO: 1, TCPPCPAPE) conjugated to the immunogen KLH.

A peptide analogueue of residues 225-233 of the human IgG1 hinge was chemically synthesized. The immunogen peptide, C-T-S-P-P-S-P-S-P-A-P-E (Cys-Thr-Ser-Pro-Pro-Ser-Pro-Ala-Pro-Glu, SEQ ID NO: 16) was designed which included an N-terminal Cys for linkage and an internal substitution of Ser for the Cys that oocurs at position 226 and 229 in the IgG to avoid oxidation and dimerization of peptides at those positions.

The peptide was prepared on an ABI 433A Peptide Synthesizer using 0.25 mmol scale FastMoc chemistry and Fmoc-Glu(OtBu)-Wang Resin. Approximately 190 mg of crude peptide was released from the resin and was purified via multiple injections onto two Vydac C-18 columns Fractions were collected and analyzed by RP-HPLC and MALDI-TOF MS. Pooled fractions yielded 38 mg of lyophilized product having free cysteine by Elliman's test.

The conjugation of the peptide to keyhole limpet hemocyanin (KLH) was accomplished with EDC coupling chemistry (1-Ethyl-3-[3-dimethylaminopropul]carbodiimide) using the Imject Immunogen EDC Kit (Pierce product no. 77622). 22 mg of purified peptide at 4 mg/mL in EDC conjugation buffer was combined with an equivalent mass of KLH at 10 mg/mL in water. Linkage of peptide to KLH occurred following addition of EDC solution to the mixture with gentle mixing for 2 hours at room temperature. All of the above steps were performed according to the manufacturer's directions. The conjugation mixture was dialyzed against PBS to remove excess linkage reagents. The KLH-peptide conjugation was confirmed by sandwich immunoassay using the polyclonal rabbit cleavage site specific specific antibody preparation RAH (reactive to a cleavage site specific analogueue peptide ending with the sequence C-P-P-C-P-A-P-E) as described in Example 1 and biotinylated anti-KLH antibody as capture and detection antibodies, respectively.

Following immunization, the reactivity of rabbit serum to the peptide analogueue was tested by ELISA. Table 4 shows the titers (as the reciprocal of the dilution at which a signal was detectable), which indicate that all of the rabbits had antibodies reactive to the cleavage site specific analogueue peptide detectable in the serum. One animal (657) had a very low level of detectable antibodies to the immunogen with a 1/titer equaling 640. Cleavage site specific antibodies were also detected in the wiffle ball fluids of all six rabbits, with the same animal (657) having a low level of detectible antibodies. Therefore, five of the six rabbits demonstrated robust cleavage site specific antibody titers detectable in both the serum and wiffle ball fluids.

TABLE 4

| Rabbit | Serum (titer) | Wiffle ball (titer) |
|---|---|---|
| 651 | 12,800 | 3,200 |
| 653 | 51,000 | 12,800 |
| 656 | 820,000 | 51,000 |
| 657 | 640 | 320 |
| 659 | 51,000 | 12,800 |
| 661 | 205,000 | 12,800 |

In the final phase of the study, the immunized and non-immunized rabbits were inoculated with 6.2 log10 colony forming units (CFUs) of S. aureus (ATCC 29213) directly into the wiffle-ball compartments.

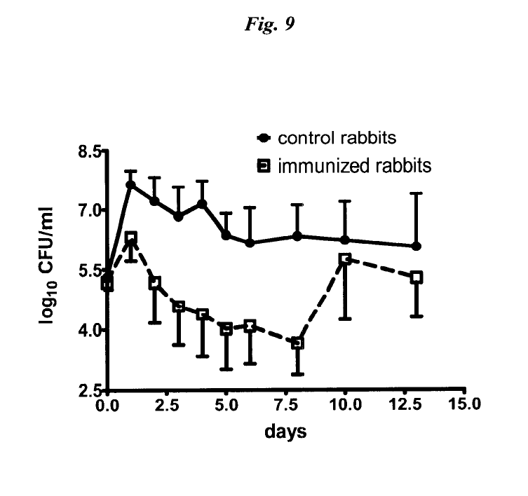

The bacterial counts and test subject viability were monitored over the course of two weeks for bacterial counts and survival. The data shown in FIG. 9 indicate that during the first week of the study, the average bacterial counts for control animals were several logs higher than cleavage site specific vaccinated animals. For example, on day 2 the control had 7.2 log₁₀ CFU/ml wiffle ball fluid while the immunized group had 5.2 log₁₀ CFU/ml wiffle ball fluid.

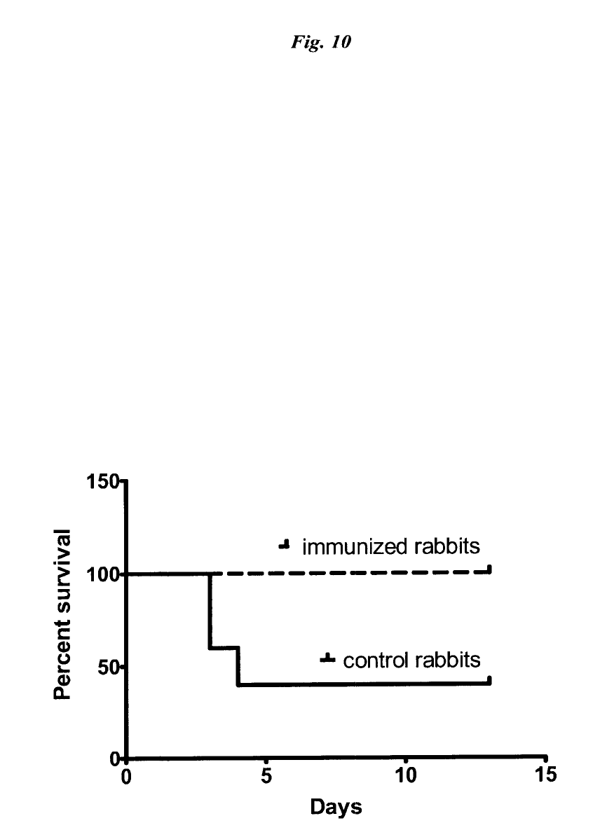

The differences in bacterial counts translated into a profound difference in rabbit viability. By the end of the two week study, only 40% percent of the control animals survived, while 100% of the cleavage site specific vaccinated animals were viable (FIG. 10).

Therefore, these results supported the hypothesis that innate immunity to pathogens, when present, can be restored to an effective level by the present method of immunization against the byproduct of pathogen produced proteases, presumably the cleaved but still immunoreactive anti-Staph. aureaus IgG. This process was evidenced by not only reducing bacterial counts of inoculated animals with a highly proteolytic bacterium, but also by the fact that the animals were rescued from mortality.

### EXAMPLE 4

### Passive Immunization to Treat Cancer

To demonstrate the principle of restoration of effector functions by an cleavage site specific antibody to a tumor targeting Mab, a murine human tumor xenograft model, the MDA MB 231 orthotopic xenograft model in SCID Beige mice, was used with an antibody called CNTO860 (U.S. Pat. No. 7,605,235), directed against human tissue factor (CD142) previously demonstrated to show the ability to reduce tumor growth in the model.

The study was designed to show that proteolytic cleavage of the lower hinge domain of the IgG1 of CNTO860 would render the antibody ineffective and evaluate if an cleavage site specific cleavage site mAb would restore the anti-tumor efficacy of cleaved CNTO860 in the MDA-MB-231 orthotopic xenograft model in SCID Beige mice.

The intact anti-tumor antigen antibody, CNTO 860, comprises a human IgG1 constant regions. A protease cleaved antibody, CNTO860, was prepared using the bacterial protease IdeS, as described above.

The antibody preparations were diluted fresh each week and supplied at 10 μg/mL in PBS for animal dosing at 0.1 mg/kg. The mAb 2095-2 (chimeric cleavage site specific cleavage_{IdeS}) was administered alone at same schedule as for the 860 variants above at 1.0 mg/kg.

Immunocompromised mice (SCID Beige mice (C.B-17/IcrCrl-scid-bgBR) approximately 18-20 g in weight obtained from Charles River Laboratories) were anesthetized and implanted with MDA-MB-231 a human breast carcinoma line (ATCC #HTB-26) cells suspended at 5×10⁷ cells/mL in serum-free DMEM into the (Right axillary number 2 or 3) mammary fat pad in a volume of 50 μL as specified by an IACUC approved protocol.

Mice, eight per group, received test substances or PBS (control group) as shown in the Table 5 (below). Group 4 received CNTO 860 single cleaved IgG dosed as specified above for mono-therapy plus mAb 2095-2 administered at 1.0 mg/kg, dosed with a 2-hr delay i.p. The dosing began on day 3 of the study and was repeated once per week.

TABLE 5

| | First Injection | | Second Injection | |
|---|---|---|---|---|
| Group | (i.v., 0.2 cc/20 g) | Dosage | (i.p.) | Dosage |
| 1 | PBS-- | — | — | |
| 2 | CNTO860 | 0.1 mg/kg | — | |
| 3 | CNTO860 single | 0.1 mg/kg | — | |
| | cleaved IgG | | | |
| 4 | CNTO860 single | 0.1 mg/kg | Cleavage site | 1 mg/kg |
| | cleaved IgG | | specific mAb | |
| | | | 2095-2 | |
| 5 | PBS | — | Cleavage site | 1 mg/kg |
| | | | specific mAb | |
| | | | 2095-2 | |

The study was terminated when the control group tumors reach 800 mm3 in volume. At termination, whole blood was collected via cardiac puncture into prepared EDTA coated tubes from all animals. At the termination of the study, body weights and tumor measurements were recorded and the primary tumors surgically removed, and weighed.

### Results

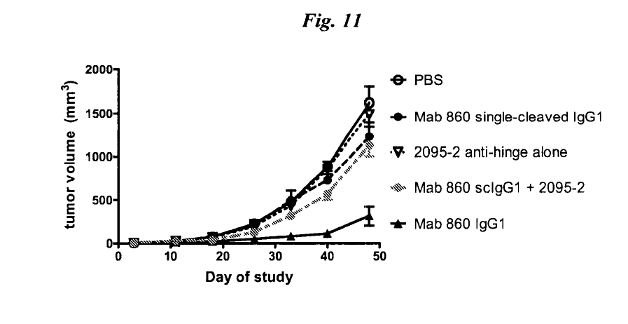

The mean tumor volumes for each group of mice at specific times during the experiment are shown in FIG. 11. Statistically, the tumor volumes in the CNTO860 treated group were lower compared to each of the other groups starting at Study Day 11. The tumor volumes in the combination CNTO860 plus mAb2095-2 group were lower than those in the PBS group from Study Days 18-40; lower than those in the CNTO860 scIgG alone group from Study Days 26-40; and lower than those in the mAb2095-2 alone group from Study Days 18-40. There were no other differences in tumor volume among the treatment groups.

The CNTO860 alone group had much smaller final tumor weights than every other group. Additionally, the combination CNTO860 plus mAb2095-2 group had smaller tumors than both the PBS and mAb2095-2 alone groups. There were no other differences among the treatment groups in tumor weight.

These results demonstrate that passive immunization with a cleavage site specific antibody preparation restores anti-tumor activity where a cleaved target specific antibody in present.

### EXAMPLE 5

### Target Cell Depletion Using a Cleavage Site Specific Antibody and Cleaved Targeting Antibody

In the following experiment, an in vivo administration of an cleavage site specific antibody (rabbit-human chimeric mAb mAb 2095-2 with specificity to human IgG1 cleaved by IdeS (chimeric cleavage site specific cleavage_{Ides})) following administration of a single-cleaved IgG or double-cleaved IgG, a F(ab′)₂, was investigated.

The anti-platelet integrin (α_{IIb}β_{IIIa}, also called IIb/IIIa) binding antibody, c7E3, was used as the targeting antibody for several reasons. The anti-platelet mAb was a human IgG1 chimeric that was shown in a pilot study to result in profound platelet clearance after 24 hours with recovery over 5 days (indicating substantial acute recognition of the mAb by the canine Fcγ receptors and/or complement system). Additionally, the effect was distinguishable at a dose of the intact IgG (0.05 mg/kg) that did not cause inhibition of platelet function and therefore not likely to cause clinically relevant bleeding complications. Lastly, circulating platelet numbers provide a readily quantifiable endpoint and the multiple blood samplings in this non-terminal study did not pose blood loss artifacts or risks for animals of this size.

### Materials and Methods

Three preparations of c7E3 were used: the intact murine-human IgG1 chimeric antibody, a single cleaved and a F(ab′)₂ prepared using the bacterial enzyme IdeS.

First, the c7E3 antibody antigen binding and platelet depletion parameters were established in dogs. The results of the test infusions of showed that 0.01 mg/kg dose (that would be calculated to be more than sufficient to drive platelet clearance in humans) was ineffective in dogs at 2 and 24 hours and was indistinguishable from the saline group group. The 0.05 mg/kg dose had little effect by 2 hours but at 24 hours had resulted in >90% platelet clearance. The 0.2 mg/kg dose of c7E3 IgG resulted in profound platelet clearance at 2 hours that was then maintained at 24 hours. These results provided the needed information to assign the minimum dose in dogs (0.05 mg/kg) that would result in substantial, acute platelet clearance (24 hours).

### Effect on Platelet Function

c7E3 inhibits platelet aggregation by binding to platelet surface IIb/III3 receptors—thereby blocking the ability of fibrinogen to bind to the receptors on activated platelets and to clump them together. The respective inhibitory profiles of intact and single-cleaved c7E3 IgG were tested in a platelet aggregation assay. Platelet aggregation is measured as an increase of light transmission through a platelet suspension after activation with a physiological agonist. The inhibition is compared to control aggregation to 5 μM adenosine diphosphate (ADP). The percent inhibition was calculated as: control aggregation—test aggregation X 100%, divided by control aggregation.

The results indicated that there was no loss of binding/inhibition of c7E3 to platelets after single lower hinge cleavage by the IdeS protease.

c7E3 IgG and c7E3 IgG single-cleaved by IdeS protease were also compared in vivo. Three groups of 5 animals received either saline control, intact c7E3 IgG (0.05 mg/kg) or c7E3 single-cleaved IgG (0.05 mg/kg). Platelet counts were monitored at baseline and at 2, 24, 48, 72 and 96 hours post-administration. Limiting the study to 5 days minimized the likelihood of anti-c7E3 immune responses that might confound the interpretation of the results. The results are presented in FIG. 3 as the mean platelet count±SD for each animal group at each time point. The extent of the error bars connotes the normal variability of platelet counts in different animals (for example, the predose counts for the 5 animals in the single-cleaved IgG treatment group were 175,000, 355,000, 305,000, 276,000 and 334,000 per μL, respectively). Nevertheless, the mean results clearly confirmed that intact c7E3 IgG induced a substantial decrease of platelet numbers with a nadir apparent at 24 hours. The single cleaved version of c7E3 IgG at the same 0.05 mg/kg dose was essentially without effect. The single-cleaved c7E3 and saline control groups showed similar profiles throughout the 96-hour monitoring period. The platelet count declines at the final 96-hour determination in both the saline control group and single-cleaved c7E3 group suggested a non-immune-mediated cause (e.g. blood volume losses from the repeated sampling).

Platelet counts in the intact c7E3 IgG group showed a gradual recovery reaching approximately 50% of the pre-dose level by 96 hours. No adverse clinical findings (e.g. bleeding) were noted in any group and this is consistent with the known human tolerance for limited periods of low circulating platelet counts and/or profound inhibition of platelets.

The finding in this study was that single proteolytic cleavage of IgG in the lower hinge results in profound loss of effector function. Despite its equivalent binding to platelets, the single cleaved derivative of c7E3 was unable to engage the components of the immune system that normally remove opsonized cells (as seen with intact c7E3 in this study). These in vivo results confirmed similar in vitro findings with several cell-targeting mAbs whose single-cleaved derivatives essentially lose all effector functions in ADCC and complement assays.

To be consistent with the prior experiments, the variants of c7E3 were all administered at the 0.05 mg/kg dose. The study protocol included 7 different groups of 3 animals as listed in Table 6. The unique aspect of the experiment (groups 6 and 7) involves the infusion of the first targeting mAb followed by the cleavage site specific mAb after a 10 minute delay. The cleavage site specific IdeS cleavage site mAb, 2095-2, was administered at 0.5 mg/kg; 10-fold higher than the dose used for all of the c7E3 variants. The higher dose was chosen in order to maximize the complexation of cleavage site specific mAb with platelet-bound c7E3 variants. All mAbs were delivered by slow infusion over a 20 minute period. Platelet counts were monitored over a 96-hour period. The platelet counts were normalized to the pre-dose count within each animal for clarity of presentation and to minimize the normal variability in numerical platelet counts in these small 3-animal groups.

TABLE 6
Study protocol for restoration of function study in dogs.

| | | | | | Blood |
|---|---|---|---|---|---|
| | | Duration | | Duration | collection |
| | Test | of 1^{st} | | of 2nd | times (H |
| Group | article(s) | infusion | Delay | infusion | after start) |
| 1 | Normal saline | 20 min | NA | NA | Predose, 2, |
| | for injection | | | | 24, 48, |
| | | | | | 72, 96 |
| 2 | c7E3 IgG @ | 20 min | NA | NA | Predose, 2, |
| | 0.05 mg/kg | | | | 24, 48, |
| | | | | | 72, 96 |
| 3 | c7E3 F(ab′)₂ @ | 20 min | NA | NA | Predose, 2, |
| | 0.05 mg/kg | | | | 24, 48, |
| | | | | | 72, 96 |
| 4 | c7E3 sc IgG @ | 20 min | NA | NA | Predose, 2, |
| | 0.05 mg/kg | | | | 24, 48, |
| | | | | | 72, 96 |
| 5 | mAb 2095-2 @ | 20 min | NA | NA | Predose, 2, |
| | 0.5 mg/mg | | | | 24, 48, |
| | | | | | 72, 96 |
| 6 | c7E3 F(ab′)₂ @ | 20 min | 10 min | 20 min | Predose, 2, |
| | 0.05 mg/kg + | | | | 24, 48, |
| | mAb 2095-2 @ | | | | 72, 96 |
| | 0.5 mg/mg | | | | |
| 7 | c7E3 sc IgG @ | 20 min | 10 min | 20 min | Predose, 2, |
| | 0.05 mg/kg + | | | | 24, 48, |
| | mAb 2095-2 @ | | | | 72, 96 |
| | 0.5 mg/mg | | | | |

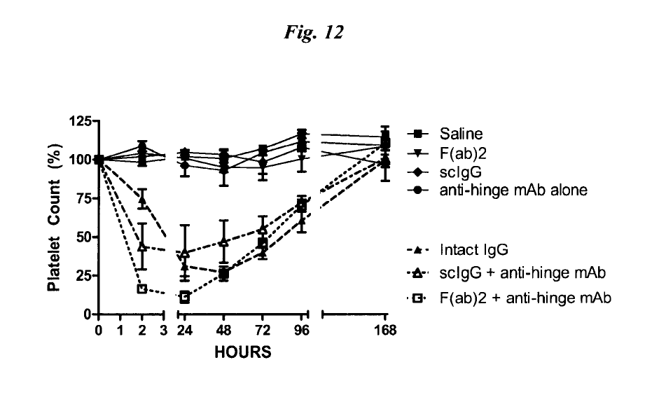

The results are presented in FIG. 12. A number of findings emerged from this study. As the the dose finding studies, intact c7E3 IgG administered at 0.05 mg/kg, induced substantial platelet clearance with a nadir apparent at 24-48 hours. Recovery of platelet numbers began at approximately 72 hours and slowly increased through the 96-hour determination. An additional blood sample was incorporated into the study at 7 days (168 hours) to allow an assessment if full recovery would occur. Full recovery did occur in all applicable treatment groups by 7 days.

In contrast to intact c7E3 IgG, single-cleaved IgG and F(ab′)₂ were without effect on platelet numbers and the platelet numbere change in these groups was not different from the saline control group. Likewise the cleavage site specific mAb, 2095-2, when infused alone (group 5) had no measurable impact on circulating platelets. However, in the c7E3 F(ab′)₂ plus mAb 2095-2 group, there occurred a rapid decrease of the platelet count such that at 2 hours the effect was nearly maximal. In addition, the extent of the platelet decline appeared to be greater in the groups where the cleavage site specific antibody was administered after the cleaved antibody fractions than in the group receiving intact c7E3 IgG (alone; group 2) at the 2 and 24 hour determinations. Thereafter, platelet counts gradually recovered and by 48 hours the rate of recovery paralleled that in the intact c7E3 IgG group.

In the F(ab′)₂ combination group, when mAb 2095-2 was coupled with single-cleaved c7E3 IgG the extent of platelet clearance was not as marked as seen in the F(ab′)₂ group and may be attributable to one poorly responsive animal in the scIgG group (also reflected in the wide error bars for this group). Nevertheless, platelet clearance occurred more quickly than had been evident in the intact c7E3 IgG group. The scIgG result is important since the targeting of this derivative with the mAb in vivo appears to have been as effective as the targeting of F(ab′)₂—an outcome that was not predictable based on in vitro findings.

### Discussion/Conclusions

The series of animal studies described here confirmed earlier in vitro findings that single-cleaved proteolytic derivatives of IgGs lose the ability to remove cells to which they are bound—in this case in vivo. The intact c7E3 IgG was shown to induce platelet clearance within 24 hours in the dog at doses >0.05 mg/kg. The 0.05 mg/kg dose was calculated to be sufficient to provide several thousand copies of IgG per platelet or about 5% α_{II}β₃ receptor blockade. A substantially higher level of receptor blockade is known to be required for inhibition of platelet function (about 80,000 copies per platelet). Thus, the canine immune system has the capacity to clear cells that are opsonized with IgG at this fractional level.

The results showed that a single proteolytic cleavage of the human IgG1 lower hinge abrogated the platelet clearance properties of the c7E3 IgG. Here, the functionally-inactive, single-cleaved IgG was prepared ex vivo by partial digestion with IdeS protease, however, and as demonstrated herein, other proteases yield IgGs with similar loss of function. The single IdeS cleavage did not impact the antigen binding characteristics of c7E3 as shown in an in vitro platelet inhibition assay. The findings suggested a mechanism to explain certain shortcomings of host immune systems in pathologies associated with hostile proteolytic environments (e.g. certain tumors, bacterial infections, inflammatory settings).

The study findings pointed to a means to correct the proteolytic defect in IgGs. Namely, a specific mAb vs. the site of proteolytic cleavage should deposit a functional Fc domain on the inactive cell-bound antibody and thereby restore Fc-mediated effector functions.

A monoclonal antibody to the IdeS cleavage site in the IgG hinge was generated by immunization of rabbits. This mAb, 2095-2, after modification to incorporate human constant regions, readily restored in vitro cell killing to inactive F(ab′)₂ or single-cleaved IgGs in ADCC and complement assays.

The key test was to employ 2095-2 in combination with IdeS-generated proteolytic derivatives of c7E3 IgG in vivo. The 2-stage protocol was designed to allow c7E3 to first bind to platelets and to then follow with an excess of the cleavage site specific mAb to complex with the platelet bound c7E3s. In the presence of coupled anti-cleaved hinge Mab and a cleaved Mab, platelet clearance was more rapid and the degree of acute platelet clearance more profound (with c7E3 F(ab′)₂) than was induced with intact c7E3 IgG alone. The rapidity of platelet clearance with the combined therapies is suggestive of an enhanced mode of immune cell killing/removal.

### EXAMPLE 6

### Cleavage Site Specific Vaccination to Treat Cancer

The use of a hinge analogueue peptide vaccination has potential to treat chronic human diseases such as invasive and metastatic cancer. Several invasive cancer animal models currently exist which are suitable for testing the efficacy of cleavage site specific directed vaccination as a means to treat invasive cancer.

The vaccination approach would confer a markedly different pharmacodynamic profile compared to anti-tumor mAb therapy alone. In the latter, an established tumor is subjected to an abrupt dosage of a monoclonal antibody to a specific target on its surface. The tumor may, or may not, already be targeted by host antibodies to the same antigen, or to additional antigens, on those cells. The vaccination strategy, if initiated early, would provide a continuous and comparatively invariant level of cleavage site specific host antibodies to engage any tumor-surface cleaved antibodies present at the sites of tumor growth or invasion as generated.

As demonstrated by using the innate humoral response of the rabbit in the previously described infection model, the cellular target of the primary response is almost immaterial; the secondary antibody wave restores effector function to any damaged primary antibody that is present. The inactivated and cell-bound-bound host antibodies would provide a variety of antigenic targets (and surface locations) for cleavage site specific Abs.

As demonstrated herein, a single- or double-cleaved antibody directed to and engaged with a cell surface antigen can be targeted by a cleavage site-specific antibody, thereby restoring the host ability to eliminate the target cell using effector functions provided by the intact cleavage site specific antibody. Another and markedly different approach would be to immunize the host against the sites of cleavage damage induced by proteases associated with specific diseases such as metastatic cancer. For example, tumor cells elaborate matrix metalloproteases that cleave IgG in the hinge. Vaccination against the sites of cleavage could provide host antibodies that would combat the molecular defects and augment immune function in that local environment.

The target of the damaged mAb(s) could be specific or could be multiply-targeted using a mixture of cleavage site specific peptides in order to protect against the action of multiple proteolytic enzymes without the need for detailed identification.

Thus, in accordance with the invention, a subject cancer patient in need of treatment, is vaccinated with a cleavage site peptide immunogen to generate a cleavage site specific antibody response either before, after or concurrent with treatment within a cancer treating antibody composition. In one instance, the immunogen is a MMP-3 or MMP-12 cleavage site peptide having the sequence terminating in the amino acid sequence P-C-P-A-P which is residues 10 to 14 of SEQ ID NO: 1.

1. A method of protecting a human subject from a pathological condition characterized by the release of a protease, wherein the method comprises administering a peptide immunogen, wherein the peptide is capable of generating antibodies reactive with a proteolytic cleavage site on IgG.

2. The method of claim 1, wherein the pathological condition is a disease selected from an arthritic disease, a malignant disease, the presence of an infectious agent or a parasite, and a vascular disease.

3. The method of claim 2, wherein the disease is rheumatoid arthritis.

4. The method of claim 2, wherein the disease is a Stapholycoccus spp. Infection.

5. The method of claim 2, wherein the disease is breast cancer.

6. The method of claim 2, wherein the subject has been diagnosed or suspected of having had a myocardial infarction or is suffering from congestive heart failure.

7. The method of claim 1, wherein the peptide represents a protease specific cleavage site in human IgG produced by a protease selected from the group consisting of a MMP-3, MMP-7, MMP-12, HNE, plasmin, cathepsin G, pepsin, IdeS, or glutamyl endopeptidase I from Staph. aureus.

8. The method of claim 7, wherein the protease specific cleavage site in a human IgG has been detected in a sample from the patient using an antibody having specificity for the protease specific cleavage site.

9. A method of treating a patient, wherein the patient is being treated with a specific antibody-containing composition, wherein antibody is susceptible to cleavage by one or more proteases associated with the specific disease being treated, the method including the step of administering to the patient a peptide immunogen wherein the peptide is a peptide fragment or analogue representative of a proteolytic cleavage site on IgG produced by the one or more proteases associated with the specific disease being treated by antibody therapy.

10. The method of claim 9, wherein the pathological condition is a disease selected from an arthritic disease, a malignant disease, the presence of an infectious agent or a parasite, and a vascular disease.

11. The method of claim 10, wherein the disease is rheumatoid arthritis.

12. The method of claim 10, wherein the disease is a Stapholycoccus spp. Infection.

13. The method of claim 10, wherein the disease is breast cancer.

14. The method of claim 10, wherein the subject has been diagnosed or suspected of having had a myocardial infarction or is suffering from congestive heart failure.

15. The method of claim 9, wherein the peptide represents a protease specific cleavage site in human IgG1 produced by a protease selected from the group consisting of a MMP-3, MMP-7, MMP-12, HNE, plasmin, cathepsin G, pepsin, IdeS, or glutamyl endopeptidase I from Staph. aureus.

16. The method of claim 9, wherein the protease specific cleavage site in a human IgG has been detected in a sample from the patient using an antibody having specificity for the protease specific cleavage site.

17. The method of claim 9, wherein the administration of the peptide immunogen is capable of restoring, enhancing or maintaining effector function of the antibody containing composition.

18. The method of claim 1 or 9, wherein the cleavage product analogue peptide immunogen is prepared by:
identifying the residue of a pair of residues of a heavy chain of an antibody cleaved by a protease most proximal to the N-terminus of the heavy chain;
identifying a peptide sequence comprising at least 5 contiguous amino acid residues which are upstream from the protease cleavage site where the residue of the protease cleavage site that is most proximal to the N-terminus of the polypeptide will become the C-terminus of the defined sequence; and
creating a solution of the peptide or peptide homolog or chemical homolog in sufficient amounts for the immunization.

19. The method of claim 1 or 9, wherein the peptide immunogen comprises at least 5 contiguous amino acids selected from the human IgG hinge region sequences of SEQ ID NO: 1, 2, 3, or 4 that are upstream from the amino terminal side of a protease cleavage site or a species homolog or chemical analogue.

20. The method of claim 1 or 9, wherein the peptide immunogen comprises at least the hinge core of IgG1, defined as the residues -T-C-P-P-C- (residues 7-11 of SEQ ID NO: 1) or a species homolog or chemical analogue.

21. The method of claim 1 or 9, wherein the peptide immunogen comprises a 12-mer peptide analogue of the human IgG1 lower hinge and adjoining CH2 domain having the sequence TCPPCPAPELLG (residues 7-18 of SEQ ID NO: 1).

22. The method of claim 1 or 9, wherein the peptide immunogen comprises a peptide having an amino acid sequence selected from SEQ ID NO: 5-11 and N-terminal truncations thereof, comprising at least 5 amino acids and containing amino acid sequences that are upstream of the N-terminal side of an IgG protease cleavage site.

23. The method of claim 1 or 9, wherein the peptide immunogen comprises an amino acid sequence selected from (a) the sequence of amino acids on the amino terminal side of an IgG1 MMP-3 cleavage site (TCPPCPAP, residues 7-14 of SEQ ID NO: 1), (b) the glutamyl endopeptidase IgG1 cleavage site (TCPPCPAPE, residues 7-15 of SEQ ID NO: 1); and (c) a peptide comprising the IdeS IgG1 cleavage site (TCPPCPAPELLG, residues 7-18 of SEQ ID NO: 1).

24. The method of claim 19, wherein the peptide is a chemical homolog, wherein the cysteine residues are replaced with serine residues.

25. The method of claim 19 wherein the peptide is conjugated to a carrier protein in a manner such that the C-terminus of the peptide is chemically bound to another moiety.

26. The method of claim 25, wherein the carrier protein is selected from the group consisting of keyhole limpet hemocyanin (KLH) or serum albumin.

27. The method of claim 19, wherein the peptide is selected from those of SEQ ID NOS 5-16.

28. A vaccine composition comprising a peptide comprising at least 5 contiguous amino acids selected from the human IgG hinge region sequences of SEQ ID NO: 1, 2, 3, or 4 that are upstream from the amino terminal side of a protease cleavage site or a species homolog or chemical analogue thereof in a pharmaceutically acceptable carrier compatible with administration to a patient by a specified route of administration.

29. A vaccine preparation of claim 28, wherein the peptide is combined with an adjuvant for the purpose of enhancing the humoral response to the peptide.

30. The vaccine preparation of claim 29, wherein the adjuvant is selected from the group consisting of CD40 agonists, alum, polymeric microparticles, an oil-in-water emulsion, MF59, CpG, GM-CSF, IL-12 and IL-2.

31. The vaccine composition of claim 28, 29 or 30 wherein the peptide is selected from those of SEQ ID NOS. 5-16.

32. A method for enhancing or maintaining effector function of an antibody administered to a patient for the treatment of a pathological condition, wherein the antibody is subject to cleavage by one or more proteases in the patient, which method comprises vaccinating the patient before, after or concurrently with the antibody treatment, with a human IgG protease cleavage site peptide capable of generating antibodies that bind to the treatment antibody and can restore effector function thereto.

33. The method of claim 32 wherein the human IgG protease cleavage site peptide used for vaccinating the patient is a peptide selected from those of SEQ ID. NOS: 5-16.


SEQUENCE LISTING (16 sequences)

SEQ ID NO: 1 - protein, 25 aa, Homo sapiens
SCDKTHTCPPCPAPELLGGPSVFLF

SEQ ID NO: 2 - protein, 41 aa, Homo Sapiens
TCNVDHKPSNTKVDKRVESKYGPPCPSCPAPEFLGGPSVFL

SEQ ID NO: 3 - protein, 41 aa, Homo sapiens
TCNVDHKPSNTKVDKTVERKCCVECPPCPAPPVAGPSVFLF

SEQ ID NO: 4 - protein, 40 aa, Homo sapiens
SCDTPPPCPRCPEPKSCDTPPPCPRCPAPELLGGPSVFLF

SEQ ID NO: 5 - protein, 14 aa, Homo sapiens
SCDKTHTCPPCPAP

SEQ ID NO: 6 - protein, 14 aa, Homo sapiens
CDKTHTCPPCPAPE

SEQ ID NO: 7 - protein, 14 aa, Homo sapiens
THTCPPCPAPELLG

SEQ ID NO: 8 - protein, 14 aa, Homo sapiens
TKVDKKVEPKSCDK

SEQ ID NO: 9 - protein, 14 aa, Homo sapiens
KVDKKVEPKSCDKT

SEQ ID NO: 10 - protein, 14 aa, Homo sapiens
DKTHTCPPCPAPEL

SEQ ID NO: 11 - protein, 14 aa, Homo sapiens
VDKKVEPKSCDKTH

SEQ ID NO: 12 - protein, 12 aa, Artificial sequence
  chemical analogue of human IgG1 peptide sequence
TAPPAPAPELLG

SEQ ID NO: 13 - protein, 12 aa, Artificial Sequence
  Serine subsituted IdeS cleavage site specific peptide
TSPPSPAPELLG

SEQ ID NO: 14 - protein, 12 aa, Artificial sequence
  Position 4 substitute human IgG1 cleavage fragment
TSPPSPAPALLG

SEQ ID NO: 15 - protein, 12 aa, Artificial Sequence
  Position 3 and 4 substitute human IgG1 cleavage fragment
TSPPSPAPEALG

SEQ ID NO: 16 - protein, 12 aa, Artificial sequence
  Peptide immunogen based on Strep pyrognes cleavage of human IgG1
CTSPPSPSPAPE